(19) United States
(12) Patent Application Publication
(10) Pub. No.: US 2025/0380888 A1
(43) Pub. Date: Dec. 18, 2025

(54) MULTIPLEXING SENSOR APPARATUS AND METHODS OF PRODUCTION THEREOF

(71) Applicant: NUTROMICS TECHNOLOGY PTY LTD, BRUNSWICK,VICTORIA (AU)

(72) Inventors: MIKEL FRASER LARSON, BRUNSWICK, VICTORIA (AU); GARRY CHAMBERS, BRUNSWICK, VICTORIA (AU); ALASTAIR MCINDOE HODGES, BRUNSWICK, VICTORIA (AU); VINICIUS ROMERO GONCALES, BRUNSWICK, VICTORIA (AU); LOUKAS TSIGARAS, BRUNSWICK, VICTORIA (AU); ARTHUR SHI PIN CHIEW, BRUNSWICK, VICTORIA (AU)

(21) Appl. No.: 18/878,381

(22) PCT Filed: Jun. 22, 2023
(86) PCT No.: PCT/AU2023/050568

§ 371 Date: Dec. 23, 2024

Related U.S. Application Data
(60) Provisional application No. 63/355,702, filed on Jun. 27, 2022.
(60) Provisional application No. 63/463,918, filed on May 4, 2023.

(30) Foreign Application Priority Data
May 12, 2023 (WO) PCT/AU2023/050401

(51) Int. Cl. A61B 5/145; A61B 5/1473

(52) U.S. Cl. CPC A61B 5/14546; A61B 5/1473

(57) ABSTRACT

An electrochemical sensor apparatus for introducing a needle electrode into the skin of a subject to contact a biological fluid or a tissue in the subject and detect a target analyte, and methods of producing the apparatus. The apparatus has a movable portion which urges the needle electrode in the subject's skin and at least two needle electrodes are selected from a library to provide the apparatus with a multiplexing capacity.

### FIELD OF THE INVENTION

The present invention relates generally to a sensor apparatus used in the detection of analytes in a test sample or in vivo. In particular, the invention relates to a sensor apparatus produced by assembling a plurality of individual electrodes. The electrodes may be selected to include two or more working electrodes, each of which is capable of specifically detecting different analytes or the same analyte.

### BACKGROUND TO THE INVENTION

Electrochemical aptamer-based (EAB) sensors open the possibility for in situ real-time monitoring of a target analyte in a subject. In that context, an EAB sensor may comprise a working electrode in the form of a needle coated with a redox-modified aptamer capable of specifically binding to a target analyte. The working electrode may be inserted into the skin of the subject such that the aptamer contacts a biological fluid such as the interstitial fluid or blood. An interrogating potential is applied to the working electrode (for example by square wave voltammetry) and current through the electrode is measured. The amount of measured current is used to determine an amount of analyte present in the biological fluid.

It may be desirable in a clinical scenario to contemporaneously detect multiple target analytes in a biological fluid of a subject. The amounts of multiple analytes of clinical concern in a particular disease or condition may be determined, with the results being used together to provide detailed diagnostic or prognostic information on the subject. For example, a subject having a suspected myocardial infarction may have blood assayed for a number of endogenous cardiac markers such as troponin, creatinine phosphokinase, C-reactive protein, and myoglobin.

Biological fluid may also be assayed for multiple exogenous analytes such as multiple drugs in a subject receiving combination therapy. In other circumstances, both endogenous and exogenous analytes may be contemporaneously detected. One such circumstance is where a nephrotoxic antibiotic drug is administered, and the amount of drug and an endogenous toxicity marker (such as a liver enzyme) is assayed.

Problems arise in applications where multiple analytes are to be detected in that multiple sensor apparatuses must be provided, adding to cost. In the previous example, a first sensor apparatus for the detection of the drug, and a second sensor apparatus for the detection of liver enzyme will be required, each apparatus having its own dedicated housing, electronics and power supply. Moreover, multiple sensor apparatuses must be applied to the subject's body, which is time consuming and inconvenient. Each applied sensor may also provide a target for catching on clothing and other objects in the subject's environment.

For sensor technologies capable of multi-analyte sensing, each electrode is independently functionalized or suitably prepared in situ thereby adding significantly to cost, time, and complexity in manufacturing.

As discussed above, EAB sensors may have electrodes in the form of needles or microneedles. A single microneedle typically has a length of 150 to 1500 μm, a width of 50 to 250 μm, with a tapered tip of thickness 1 to 25 μm. Microneedles may be fabricated from metal, silicon, polymer, glass, or ceramic, with the base of the microneedles typically being attached to a base substrate to form an array. The microneedle base substrate may comprise an adhesive to improve engagement with the skin.

The prior art discloses a number of apparatuses that insert microneedles into the skin of a subject. Such apparatuses are typically configured to facilitate application of microneedles by the subject in a non-clinical setting such as in the home. Ease of use and reproducibility are key aims of these apparatuses.

Some apparatuses are dedicated to the application of microneedles only, and once that task is completed, the device is removed along with the microneedles. Other prior art applicator apparatuses are configured to be separated from the microneedles, thereby allowing the microneedles to remain in situ in the skin for a period of time after introduction.

Yet a further type of prior art apparatus is configured to introduce the microneedles, with the apparatus (including the microneedles) remaining in situ on the subject for a period of time. While these apparatuses offer simplicity of use for the subject, they nevertheless present a number of problems.

One problem is that such apparatuses are generally obtrusive and are readily noticeable by the subject. The apparatus may catch on clothing or any other nearby object leading to complete or partial dislodgement. These apparatuses may need to be worn overnight, with significant discomfort arising where the subject rolls onto the apparatus.

A further problem is that prior art apparatuses are complex having a large number of individual parts. This increases cost and also the propensity for failure. A large number of parts also increases weight thereby increasing obtrusiveness for the subject. The discomfort associated with apparatus weight is found to increase proportionally with the duration for which it is worn. For some applications (such as hormone monitoring) continuous real-time data may be required over a period of weeks. While the apparatus is likely to be changed a number of times over that period, the problem of the subject wearing a weighty item for an extended period remains.

A further problem arises in that the subject may be uncertain if the microneedles have properly penetrated the skin at first instance, and furthermore whether they remain properly embedded in the skin over time. Prior art sensor apparatuses typically comprise a housing, the lower face of which sits flush on the surface of the skin. It is difficult, if not impossible, for the subject to view the surface of the skin to check for proper microneedle embedment given the presence of the housing. Where there is doubt, the apparatus may be removed and a new one applied. Replacement will be wasteful where the microneedles were in fact properly inserted.

It is an aspect of the present invention to provide an improvement to prior art sensor apparatuses and methods of production thereof. It is a further aspect of the present invention to provide a useful alternative to prior art sensor apparatuses and prior art production methods.

The discussion of documents, acts, materials, devices, articles and the like, is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, but not necessarily the broadest aspect, the present invention provides a method for producing an electrochemical aptamer-based sensor apparatus, the method comprising assembling two or more electrodes of an electrochemical aptamer-based sensor with an apparatus for contacting the two or more electrodes to the skin of a subject.

In one embodiment of the first aspect, at least one of the two or more electrodes is a working electrode configured to specifically detect an analyte.

In one embodiment of the first aspect, at least two of the two or more electrodes is each a working electrode configured to specifically detect an analyte.

In one embodiment of the first aspect, each of the two or more working electrodes comprises a different aptamer species, each of the different aptamer species configured to specifically detect different analytes or the same analyte.

In one embodiment of the first aspect, the two or more working electrodes are assembled in a fixed mutual spaced relationship.

In one embodiment of the first aspect, the method comprises assembling one or more counter electrodes with the two or more working electrodes.

In one embodiment of the first aspect, the method comprises assembling one or more reference electrodes with the two or more working electrodes.

In one embodiment of the first aspect, the two or more working electrodes, and the electrodes are regularly arranged.

In one embodiment of the first aspect, the regular arrangement is an array.

In one embodiment of the first aspect, each of the two or more working electrodes is substantially equidistant to one of the one or more counter electrodes.

In one embodiment of the first aspect, each of the two or more working electrodes is substantially equidistant to one of the one or more reference electrodes.

In one embodiment of the first aspect, all electrodes are disposed in a fixed mutual spatial relationship.

In one embodiment of the first aspect, the distance or average distance between the electrodes is less than about 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, or 0.1 mm.

In one embodiment of the first aspect, all electrodes are disposed within an area of less than about 100 mm², 90 mm², 80 mm², 70 mm², 60 mm², 50 mm², 40 mm², 30 mm², 20 mm², 10 mm², 9 mm², 8 mm², 7 mm², 6 mm², 5 mm², 4 mm², 3 mm², 2 mm², or 1 mm².

In one embodiment of the first aspect, at least one of the two or more working electrodes, and/or at least one of the one or more counter electrodes, and/or at least one of the one or more reference electrodes is/are a wire, a needle, or a microneedle.

In one embodiment of the first aspect, the assembling comprises mounting each of the electrodes on a mounting portion.

In one embodiment of the first aspect, the mounting portion is substantially resistant to flexing and/or stretching and/or contracting.

In one embodiment of the first aspect, the mounting portion electrically insulates each electrode from each other electrode.

In one embodiment of the first aspect, the electrodes and/or the mounting portion are configured to form a watertight seal at a junction formed therebetween.

In one embodiment of the first aspect, the watertight seal is formed by way of a press fit, snap fit, or friction fit, between the electrode and the mounting portion.

In one embodiment of the first aspect, the watertight seal is formed by way of a flexible seal, or a curable sealant applied to or about the junction.

In one embodiment of the first aspect, the watertight seal is formed by way of a threaded connection between the electrode and the mounting portion.

In one embodiment of the first aspect, at least one of the electrodes comprises an expanded region configured to contact a surface of the mounting portion.

In one embodiment of the first aspect, the method comprises assembling at least 3, 4, 5, 6, 7, 8, 9, 10, 20, 30, 40, 50, 60, 70, 80, 90, or 100 working electrodes.

In one embodiment of the first aspect, the electrodes are each a wire, a needle, or a microneedle.

In one embodiment of the first aspect, each of the working electrodes is obtained by removing a working electrode from a group of working electrodes of the same analyte specificity.

In one embodiment of the first aspect, the group of working electrodes are held in a holder configured to releasably hold the electrodes.

In one embodiment of the first aspect, the two or more working electrodes are selected from an electrode library comprising a plurality of working electrodes each of which comprises a different aptamer species.

In one embodiment of the first aspect, the electrodes comprising the same aptamer species are grouped into a discrete holder, or grouped into a region of a single holder.

In one embodiment of the first aspect, the apparatus for contacting the two or more electrodes to the skin of a subject comprises:
a skin contacting portion defining a skin contacting surface and one or more spaces allowing the two or more electrodes to extend therethrough; and
a movable portion configured to move the two or more electrodes from a first position behind the skin contacting surface to a second position proud of the skin contacting surface.

In one embodiment of the first aspect, the apparatus comprises a retaining portion configured to, in use, retain the skin contacting surface in contact with the skin.

In one embodiment of the first aspect, the movable portion is configured to move from the first position to the second position in a non-linear path.

In one embodiment of the first aspect, the non-linear path is a generally arcuate path.

In one embodiment of the first aspect, the movable portion has a connected end and a free end.

In one embodiment of the first aspect, the free end travels a greater distance than the connected end.

In one embodiment of the first aspect, the non-linear path is described by reference to the free end.

In one embodiment of the first aspect, the non-linear path is less than about 10 mm, 9 mm, 8 mm, 7 mm, 6 mm, 5 mm, 4 mm, or 3 mm.

In one embodiment of the first aspect, the degree measure of the arc is less than about 45°, 40°, 35°, 30°, 25°, 20°, 15°, 14°, 13°, 12°, 11°, 10°, 9°, 8°, 7°, 6°, or 5°.

In one embodiment of the first aspect, the movable portion has a pivoting portion, a hinging portion, a flexing portion, or an attaching portion.

In one embodiment of the first aspect, the movable portion is associated with a mounting portion.

In one embodiment of the first aspect, in use, the mounting portion is stationary, and the movable portion is movable relative to the mounting portion.

In one embodiment of the first aspect, the mounting portion comprises a portion allowing the movable portion to pivot, hinge, flex, or attach.

In one embodiment of the first aspect, the mounting portion is in fixed spaced relation to the skin contacting surface.

In one embodiment of the first aspect, the mounting portion is spaced less than about 10 mm, 9 mm, 8 mm, 7 mm, 6 mm, 5 mm, 4 mm, 3 mm, or 2 mm, from the skin contacting surface.

In one embodiment of the first aspect, the mounting portion is generally lateral to the movable portion.

In one embodiment of the first aspect, the apparatus further comprises a user actuatable releasing portion configured to retain the movable portion in the first position until user actuation of the releasing portion, at which time the movable portion is released and allowed to move to the second position.

In one embodiment of the first aspect, the apparatus further comprises a locking portion configured to lock the movable portion when in the second position.

In one embodiment of the first aspect, the apparatus is configured such that movement of the movable portion from the first position to the second position requires a motive force originating internal and/or external to the apparatus.

In one embodiment of the first aspect, the motive force internal to the apparatus originates from a spring, an elastically deformable member, a shape memory member, or other biasing means; and the motive force external to the apparatus originates from a user.

In one embodiment of the first aspect, the apparatus is devoid of an internal motive force generator configured to move the movable portion from the first position to the second position.

In one embodiment of the first aspect, the retaining portion is or comprises a dermatologically acceptable composition disposed on or about the skin contacting surface.

In one embodiment of the first aspect, the dermatologically acceptable composition is an adhesive or a functional equivalent thereof.

In one embodiment of the first aspect, the retaining portion is configured to mechanically retain the skin contacting surface in contact with the skin.

In one embodiment of the first aspect, the retaining portion is selected from any one or more of: a strap, a band, a belt, a clamp, a grip, a tie, a clasp, a sleeve, a stocking, a sock, a glove, a cap, a hat, an underpant, a singlet, a shirt, a brassiere, a top, a trouser, a scarf, a ring, a spectacle, and a choker.

In one embodiment of the first aspect, the two or more electrodes are mechanically connected directly or indirectly to the moving portion.

In one embodiment of the first aspect, the two or more electrodes are wire(s), needle(s), and/or microneedle(s).

In one embodiment of the first aspect, the two or more electrodes form an array.

In one embodiment of the first aspect, the two or more electrodes are of sufficient length so as to be contactable with the epidermis, the dermis, or the hypodermis of the subject.

In one embodiment of the first aspect, the two or more electrodes are configured to function, in use, so as to: conduct an electric current to or from or through the skin, conduct a sound wave to or from or through the skin, conduct light to or from or through the skin, conduct heat to or from or through the skin, sample a fluid or a tissue from the skin, or deliver a biologically active substance to the skin, or introduce an analyte sensing substance to the skin.

In one embodiment of the first aspect, the two or more electrodes are each electrically conductive and the apparatus further comprises a circuit having an audio, visual or tactile indicator, the circuit configured to actuate the indicator when the one or more projecting portion(s) are in contact with an electrically conductive fluid naturally present in the skin.

In one embodiment of the first aspect, the circuit comprises at least two projecting portions and the circuit is configured to be completed by the at least two projecting portions contacting the electrically conductive fluid naturally present in the skin so as to actuate the indicator.

In one embodiment of the first aspect, the circuit comprises one projecting portion and at least one electrically conductive pad placed against the skin and the circuit is configured to be completed by the projecting portion and the pad electrically communicating with the conductive fluid naturally present in the skin so as to actuate the indicator.

In one embodiment of the first aspect, the apparatus comprises a housing dimensioned such that when the apparatus is applied to the skin and the movable portion is in the second position and any part of each of the two or more electrodes proud of the skin contacting surface are embedded in the skin, the housing extends above the skin for most part or for substantially all part no more than about 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In one embodiment of the first aspect, the apparatus is configured for use for a period of greater than about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, or 96 hours.

In one embodiment of the first aspect, the apparatus is configured such that the two or more electrodes are inseparable, or not separable without the assistance of a tool, from the apparatus.

In one embodiment of the first aspect, the movable portion and the mounting portion are integral.

In one embodiment of the first aspect, the integral moving portion and mounting portion is fabricated from an elastically deformable material.

In one embodiment of the first aspect, the integral moving portion and mounting portion is part of a circuit board of the apparatus.

In one embodiment of the first aspect, the movable portion is biased toward the second position and maintained in the first position and against the bias by the user actuatable releasing portion until actuation of the releasing portion, at which time the movable portion is released and allowed to move to the second position.

In one embodiment of the first aspect, the user actuatable releasing portion is a ledge configured to retain the movable portion in the first position, and a motive force provided by the user deforming the ledge and/or the movable portion so as to allow the moving portion to release from the ledge and move to the second position.

In one embodiment of the first aspect, the movable portion is in hinged association with the skin contacting portion.

In one embodiment of the first aspect, the hinge is disposed at or toward a peripheral region of the movable portion and the skin contacting portion.

In one embodiment of the first aspect, the releasing portion comprises a member configured to maintain the movable portion in the first position, but is removable or deformable by the user so as to allow the movable portion to move to the second position.

In one embodiment of the first aspect, the member is removable by sliding generally across the skin contacting portion.

In one embodiment of the first aspect, the member is generally wedge-shaped, and the apparatus comprises a hinge associating the movable portion with the skin contacting portion, and the thin portion of the wedge disposed proximal to the hinge and the thick portion of the wedge disposed distal to the hinge.

In one embodiment of the first aspect, the releasing portion is removable from the apparatus and comprises a gripping portion to facilitate manual removal.

In a second aspect, the present invention provides an electrochemical aptamer-based sensor apparatus comprising an assembly of two or more electrodes with an apparatus for contacting the two or more electrodes to the skin of a subject.

In one embodiment of the second aspect, at least one of the two or more electrodes is a working electrode comprises an aptamer species configured to specifically detect an analyte.

In one embodiment of the second aspect, at least two of the two or more electrodes is a working electrode, each of the two or more working electrodes comprising a different aptamer species.

In one embodiment of the second aspect, the two or more electrodes are assembled in a fixed mutual spaced relationship.

In one embodiment of the second aspect, one of two or more electrodes is a counter electrode.

In one embodiment of the second aspect, one of the two or more electrodes is a reference electrode.

In one embodiment of the second aspect, the electrodes are regularly arranged.

In one embodiment of the second aspect, the regular arrangement is an array.

In one embodiment of the second aspect, the two or more electrodes comprise a counter electrode and two or more working electrodes, wherein each of the two or more working electrodes is substantially equidistant to the counter electrode.

In one embodiment of the second aspect, the two or more electrodes comprise a reference electrode and two or more working electrodes, wherein each of the two or more working electrodes is substantially equidistant to the reference electrode.

In one embodiment of the second aspect, all electrodes are disposed in a fixed mutual spatial relationship.

In one embodiment of the second aspect, wherein the distance or average distance between the electrodes is less than about 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, or 0.1 mm.

In one embodiment of the second aspect, all electrodes are disposed within an area of less than about 100 mm², 90 mm², 80 mm², 70 mm², 60 mm², 50 mm², 40 mm², 30 mm², 20 mm², 10 mm², 9 mm², 8 mm², 7 mm², 6 mm², 5 mm², 4 mm², 3 mm², 2 mm², or 1 mm².

In one embodiment of the second aspect, at least one of the two or more working electrodes, and/or at least one of the one or more counter electrodes, and/or at least one of the one or more reference electrodes is/are a wire, a needle, or a microneedle.

In one embodiment of the second aspect, the electrodes are mounted on a mounting portion.

In one embodiment of the second aspect, the mounting portion is substantially resistant to flexing and/or stretching and/or contracting.

In one embodiment of the second aspect, the mounting portion electrically insulates each electrode from each other electrode.

In one embodiment of the second aspect, working electrodes are formed separately from the mounting portion, the working electrodes and mounting portion being assembled to form the apparatus.

In one embodiment of the second aspect, the working electrodes and/or the mounting portion are configured to form a watertight seal at a junction formed therebetween.

In one embodiment of the second aspect, the watertight seal is formed by way of a press fit, snap fit or friction fit between the electrode and the mounting portion.

In one embodiment of the second aspect, the watertight seal is formed by way of a flexible seal, or a curable sealant applied to or about the junction.

In one embodiment of the second aspect, the watertight seal is formed by way of a threaded connection between the electrode and the mounting portion.

In one embodiment of the second aspect, at least one of the electrodes comprises an expanded region configured to contact a surface of the mounting portion.

In one embodiment of the second aspect, the apparatus comprises at least 3, 4, 5, 6, 7, 8, 9, 10, 20, 30, 40, 50, 60, 70, 80, 90, or 100 working electrodes.

In one embodiment of the second aspect, wherein the electrodes are each a wire, a needle, or a microneedle.

In one embodiment of the second aspect, wherein each of the electrodes is obtained by removing a working electrode from a group of electrodes having the same aptamer species, dimension, material or function.

In one embodiment of the second aspect, the group of electrodes is held in a holder configured to releasably hold the electrodes.

In one embodiment of the second aspect, at least one of the electrodes is a working electrode, and the working electrode(s) are selected from an electrode library comprising at least 2, 3, 4, 5, 6, 7, 8, 9, 10, 11, 12, 13, 14, 15, 16, 17, 18, 19, or 20 working electrodes each of which comprises a different aptamer species.

In one embodiment of the second aspect, working electrodes comprising the same aptamer species are grouped into a discrete holder, or grouped into a region of a single holder.

In one embodiment of the second aspect, the apparatus has a feature or features of the apparatus defined in any embodiment of the first aspect that refers to an apparatus.

In a third aspect, the present invention provides a system for producing an electrochemical aptamer-based sensor apparatus, the system comprising:
a library of two or more electrodes of an electrochemical aptamer-based sensor apparatus, and
a mounting portion configured to mount the two or more working electrodes in a fixed mutual spaced relationship,
wherein the mounting portion is provided by an apparatus having the features supra.

In one embodiment of the third aspect, the two or more electrodes are each working electrodes, each working electrode comprising a different aptamer species.

In one embodiment of the third aspect, the electrode library comprises working electrodes comprising at least 2, 3, 4, 5, 6, 7, 8, 9, 10, 11, 12, 13, 14, 15, 16, 17, 18, 19, or 20 different aptamer species.

In one embodiment of the third aspect, the electrodes comprising the same aptamer species specificity are grouped into a discrete holder, or grouped into a region of a single holder.

### BRIEF DESCRIPTION OF THE FIGURES

FIG. 1 illustrates an exemplary means for producing a customised assembly of working electrodes from an electrode library.

FIG. 2 is a flow diagram showing a range of possible combinations of treatments to different sets of microneedles electrodes. The original group of electrodes are separated through process, regrouped, and then separated again.

FIG. 3 illustrates an exemplary microneedle electrode. The dimensions are shown in mm. For the avoidance of doubt, the various dimensions and angle shown on the drawing are non-limiting on the drawn embodiment.

FIG. 4 illustrates electrode needle assembly with an over-moulded needle, needle, and insolation coating, inserted into the bottom enclosure. This may be assembled by pressing the assembly into the bottom enclosure from the bottom side, or from the top. FIG. 4B is a cross-sectional view of FIG. 4A.

FIG. 5 illustrates highly diagrammatically and in lateral view a microneedle embedding apparatus useful in the context of the present invention. The embodiment relies on a biasing means to provide a motive force for insertion of the microneedles into the skin. The arm is shown in the first position (520a), as it is presented to the user, and in the second position (520b) when the microneedles are embedded in the skin. The curvature in the movable arm is shown deliberately exaggerated to better demonstrate the operation of the embodiment as a whole. While such a curvature will be operable (and therefore not excluded from the ambit of the invention), the curvature will typically be of a materially lower magnitude.

FIG. 6A illustrates highly diagrammatically and in lateral view a further microneedle embedding apparatus of the present invention. The embodiment relies on the user to provide the motive force for insertion of the microneedles into the skin. The arm is shown in the first position (505a), as it is presented to the user, and in the second position (505b) when the microneedles are embedded in the skin.

FIG. 6B illustrates a variation of the embodiment of FIG. 6A, being devoid of an upper housing.

FIG. 7A illustrates an upper perspective view of an embodiment of the present invention that utilises a printed circuit board (PCB) as the biasing means to provide the motive force for insertion of the microneedles into the skin. The arm (520) is shown in the first position as it is presented to the user, and before embedment of the microneedles into the skin.

FIG. 7B illustrates the embodiment of FIG. 7A, but in lower perspective view.

FIG. 8 illustrates an upper perspective view a microneedle embedding apparatus of the present invention. The embodiment relies on the user to provide the motive force for insertion of the microneedles into the skin. The arm is shown in the first position as it is presented to the user, and before embedment of the microneedles in the skin.

FIG. 9A illustrates a lower perspective view of the embodiment of FIG. 8.

FIG. 9B illustrates an upper perspective view of the embodiment of FIG. 8.

FIG. 10 illustrates a lower perspective view of the embodiment of FIG. 8 more completely showing the removable flexible layer that is removed to expose the dermatologically acceptable adhesive.

FIG. 11 illustrates in lower perspective view the microneedle embedding apparatus of FIG. 8 having the removable flexible layer removed to expose the dermatologically acceptable adhesive.

FIG. 12 illustrates in lower perspective view the microneedle embedding apparatus of FIG. 11 with the microneedles in an extended position, as required for embedment in the skin of a subject.

FIG. 13 illustrates a further microneedle apparatus of the present invention comprising a temperature sensor. The apparatus is further configured to prevent the outward extension of the microneedles until the apparatus is applied to the skin surface. The central area of the drawing sheet shows the components of the apparatus in lateral view, and in exploded form. Each component is shown in perspective view in the peripheral areas of the drawing sheet.

Unless otherwise indicated herein, features of the drawings labelled with the same numeral are taken to be the same features, or at least functionally similar features, when used across different drawings.

The drawings are not prepared to any particular scale or dimension and are not presented as being a completely accurate presentation of the various embodiments.

### DETAILED DESCRIPTION OF THE INVENTION AND PREFERRED EMBODIMENTS THEREOF

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise” and variations of the word, such as “comprising” and “comprises” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

As used herein, positional terms such as “lateral”, “across”, “above”, “superior”, “below”, “higher”, “lower”, “upward”, “downward”, “plan view”, and the like, are to be considered with reference to an apparatus as used with the electrodes directed downwardly toward the ground.

The articles “a” and “an”, as used herein, refer to one or to more than one (i.e., to at least one) of the grammatical object of the article. By way of example, “an element” means one element or more than one element.

The terms “about” and “approximately”, as used herein, refer to conditions (e.g., amounts, levels, concentrations, time, etc.) that vary by as much as 20% (i.e., ±20%), especially by as much as 10%, 9%, 8%, 7%, 6%, 5%, 4%, 3%, 2% or 1% to a specified condition.

The term “and/or”, as used herein, refers to and encompasses any and all possible combinations of one or more of the associated listed items, as well as the lack of combinations when interpreted in the alternative (or).

The term “plurality” refers to more than one, such as 2 through to 1×10¹⁵ (or any integer therebetween) and upwards, including 2, 10, 100, 1000, 10000, 1×10⁶, 1×10⁷, 1×10⁸, 1×10⁹, 1×10¹⁰, 1×10¹¹, 1×10¹², 1×10¹³, 1×10¹⁴, 1×10¹⁵, etc. (and all integers therebetween).

The term “subject” is used to refer to an animal (including a human and a non-human animal) to which the present invention may be applied. The term “user” is used to refer to a human that applies the apparatus to a human or a non-human animal. The subject and the user may be the same human subject, but not necessarily so.

As used herein, a “biological fluid” may be any biological fluid of a subject, including but not limited to, interstitial fluid (ISF), blood, saliva, a lacrimal secretion, a lactational secretion, a nasal secretion, a tracheal secretion, a bronchial secretion, an alveolar secretion, a gastric secretion, a gastric content, a glandular secretion, a vaginal secretion, a uterine secretion, a prostate secretion, semen, urine, sweat, cerebrospinal fluid, a glomerular filtrate, an hepatic secretion, bile, or an exudate, any of which are contacted in use with an electrode of the invention.

Unless the contrary intention is apparent from the context of use, the terms “needle”, “microneedle” and “wire” are used interchangeably. Each is functionally the same or similar, being able to insert into the skin of a subject to contact a biological fluid.

The present invention is predicated at least in part on the inventors' discovery that a multi-analyte detecting electrochemical aptamer-based (EAB) sensor apparatus may be produced by assembling at least two working electrodes, each electrode having a different species of aptamer. The different species of aptamer may be configured to detect the same analyte, or different analytes. The mutual proximity of the assembled working electrodes allows for incorporation into a single apparatus, the proximity nevertheless allowing each working electrode to faithfully detect amounts of the respective analytes in a test sample. The proximity of the assembled working electrodes allows for manufacture of small wearable apparatuses capable of monitoring the amounts of clinically important analytes in real-time. In such wearable apparatuses, the electrodes may be wires, needles, or microneedles, which contact the ISF or any other relevant bodily material of a subject. For in vitro detection of analytes, the mutual proximity of the working electrodes allows for contact with a single sample disposed in a tube or a microplate well for example.

The assembly of individual working electrodes may be effected by the user (as distinct from a manufacturing facility) to allow for instant customisation for a particular combination of target analytes. For example, personnel in a hospital may assemble several working electrodes to provide a customised EAB sensor apparatus for a certain patient. As an example, it may be necessary to monitor a diabetic patient having a bacterial infection, for glucose, the serum concentration of the antibiotic vancomycin, and a marker of vancomycin-induced nephrotoxicity such as creatinine. Such an assembly may also be used for in vitro testing (using a blood sample in a vessel, for example) or for in vivo real-time analyte detection.

In some instances, however, the assembly of working electrodes may be undertaken in a manufacturing facility according to a custom order, or according to a predetermined group of working electrode specificities that are often used in certain applications. The assembly in that regard may be performed by human or robotic means.

In the working electrode assembly, the working electrodes may be maintained in a fixed mutual spaced relationship. A fixed mutual spaced relationship may also be provided in respect of non-working electrodes such as counter electrodes and reference electrodes. Such relationship may be achieved and maintained by mounting the electrodes on a mount of some description. In one embodiment, the mount is of unitary construction and comprises apertures, each sized to snugly receive a working electrode. In other embodiments, each working electrode is embedded in a part of a mount, and a number of parts are brought together (say, by snap fitting) to create a whole mount holding a number of different working electrodes. In yet a further embodiment, the electrodes are fixed to a stiff framework capable of maintaining the working electrodes in a fixed mutual spaced relationship. A further possibility is that the mount may be provided in liquid form, the working electrodes disposed in the liquid, and the liquid then transformed to a solid form (say, by polymerisation or drying) so as to maintain the working electrodes in a fixed mutual spaced relationship.

The mount will be typically electrically non-conducting to prevent shorting between the electrodes. Where a conducting material is used, insulation may be used to prevent shorting. The mount may have a generally planar surface. In some embodiments, the mount is rigid, semi-rigid, or at least partially rigid, for facilitating penetration of the electrodes on application to the skin. The mount may also be partially flexible or semi-flexible, so that, in wearable uses, the EAB sensor apparatus can conform to an outer surface or an outer shape of at least part of a subject's body.

The mount may be fabricated from or contain woven and non-woven fabrics including electronic fabrics; natural or synthetic fibres; natural or synthetic textiles; silk; organic materials; natural or artificial composite materials, including polymeric materials; glass; ceramics, including polymer ceramics; porous materials; polymers such as rigid or semi-rigid plastics and machinable polymers such as such as acrylic, polycarbonate, polyether ether ketone, or PEEK; synthetic polymers such as polymethyl methacrylate or acrylic glass, and other plastics made from methacrylate; thermoplastics and thermosetting plastics such as acrylic resin, polycarbonate, and polyether ether ketone; thermoplastic polymers such as polyethylene terephthalate; doped polymers such as polyacetylene, polypyrrole, polyindole, and polyaniline; intrinsically conducting polymers; metals, including aluminium, copper, gold including colloidal gold, silver including colloidal silver, chromium, platinum, titanium; metal alloys including stainless steel; carbon including colloidal carbon, carbon-nano materials, and carbon composites such as graphene and graphite; semiconductors such as silicon, germanium, and gallium arsenide; doped semiconductors; and, organosilicates.

Electrodes used in the context of the present invention can be fabricated in a range of various shapes and geometries, although their specific geometry for transdermal applications be optimised to breach the stratum corneum for reliable skin penetration. The present apparatus may be configured to be urged into the skin of a subject to facilitate the electrodes breaching the stratum corneum and to penetrate through the skin layers. For non-human applications, the stratum corneum may be replaced by an analogous, or even a non-analogous layer on the surface of the subject.

Generally, each electrode will have the shape of a protruding pointed structure extending from the mount. Typically, the electrodes will extend generally perpendicular from the mount.

The protruding structure of each electrode can be of any needle-type shape. For example, protruding structure may taper smoothly from a base to form a pointed tip (e.g., cone shape), may have multiple lateral sides extending from a base that converge to form a pointed tip (e.g., pyramid shape or triangular prism), be tapered in just one dimension, or have a base with curved sides of relatively constant diameter, which is segmented to form a pointed tip (e.g., a segment of a cylindrical shape). Typically, the pointed tip will be sharp. The electrode may or may not include shape changes along its length. Further, any edge or side of the shape may be bevelled, curved, or rounded.

In some embodiments, the shape of is a cone, or a pyramid such as a triangular pyramid, square pyramid, or hexagonal pyramid. In other embodiments, the shape of a tetrahedron or a triangular prism. In further embodiments, it may take the shape of a rocket, turret, arrowhead, spike, or spear.

It will be appreciated that a range of other shapes could be used. For example, the shape may a circular or an elliptical cylinder, which is truncated. Any of the other shapes described herein may or may not be truncated. The term “truncated”, as used in this context, may refer to a shape cut on a plane parallel to the base, which may be referred to as a parallel-truncated shape or more specifically, a frustum, or a shape cut at an angle relative to an axis of the electrode, which may be referred to as an angular-truncated shape. For angular-truncated shapes, the angle of truncation relative to an axis of the shape will be at least about 50° and no more than about 75°. In some embodiments, the truncation angle is between about 55° and about 70°, about 55° and about 65°, and about 50° and about 60°. In other embodiments, the truncation angle is about 50°, about 60°, or about 65°, or about 70°. In a particular embodiment, the shape of the electrode is a truncated circular cylinder, with a truncation angle of about 60° relative to its axis.

It will be appreciated that the same or different shapes could be provided on the mount. For example, the electrode could be shaped as a plate or blade with a sharp edge.

Electrodes contemplated by this invention can be divided generally into 4 types: solid, coated, dissolving, and hollow. However, it will be appreciated that the mount could comprise a combination of the 4 types. For example, the mount may comprise a combination of solid and hollow electrodes. For example, there may be no requirement for any to function as a counter electrode or a reference electrode to be a particular type.

A hollow electrode, for example, will generally have a hollow interior defined by an interior wall with an opening at the terminus intended to contact a biological fluid. The hollow interior may or may not conform to the outer shape of the electrode. In some embodiments, the hollow electrode has a generally circular hollow interior, like a bore hole. The circular hollow interior may have a diameter of between at least about 0.1 mm and no more than about 5 mm. In some embodiments, the diameter of the hollow interior is between about 0.5 mm and about 1 mm. The opening to the hollow interior is preferably in proximity to the terminus, but could also be at the top face of the hollow electrode.

The exterior wall of the electrode may be configured to abut against the stratum corneum of a subject to control depth of penetration into the skin layers of the subject. The exterior wall may also be provided with a shoulder or ledge for this purpose.

The exterior wall may have a smooth or rough surface, and can include surface features, such raised portions, etchings, serrations, anchors, barbs, or the like, which may assist engaging a biological tissue once the electrodes have breached the stratum corneum to secure them within the subject. It will be appreciated that the ability of an EAB sensor to remain in situ is particularly beneficial, as this ensures that continuous measurements over a prolonged period of time are made at the same site within the subject. Furthermore, constraining the location in which measurements are performed ensures more accurate longitudinal monitoring. In some embodiments, the EAB sensor is configured to remain in situ for at least one minute, at least one hour, at least about 8 hours, at least about 18 hours, at least one day (about 24 hours), at least about 3 days, at least about 4 days, or at least one week. In some applications it may be necessary or desirable to remain in situ for one month.

The exterior wall of the electrodes may or may not have void spaces. In some embodiments, the exterior wall of the hollow electrode is porous, or has a porous layer, which may increase the effective surface area of the electrode, or may allow a target of interest to enter the pores, but exclude one or more other targets or substances, depending on the size of the target of interest. The pores may be less than about 10 μm in diameter, preferably less than about 1 μm in diameter.

It will be appreciated that the size of the electrodes, and their arrangement on the mount, may vary depending upon the intended application.

The electrodes may be of a length at least greater than the thickness of the stratum corneum and to penetrate the skin layers to a depth of at least 100 μm, to be positioned in a biological tissue to contact a biological fluid of a subject. In some embodiments, the length will be at least about 10% greater than the thickness of the stratum corneum, at least about 20% greater than the thickness of the stratum corneum, at least about 50% greater than the thickness of the stratum corneum, at least about 75% greater than the thickness of the stratum corneum, or at least about 100% greater than the thickness of the stratum corneum. In some embodiments, the length is less than about 1500 μm, less than about 1000 μm, less than about 750 μm, less than about 600 μm, less than about 500 μm, less than about 400 μm, less than about 300 μm, less than about 250 μm, greater than about 100 μm, greater than about 50 μm, greater than about 20 μm, or greater than about 10 μm. In other embodiments, the length is between about 100 μm and about 1000 μm, about 200 μm and about 1000 μm, about 500 μm and about 1000 μm, about 750 μm and about 1000 μm, about 800 μm and about 1000 μm, about 900 μm and about 1000 μm, about 100 μm and about 900 μm, about 200 μm and about 900 μm, about 500 μm and about 900 μm, about 750 μm and about 900 μm, about 800 μm and about 900 μm, about 100 μm and about 800 μm, about 200 μm and about 800 μm, about 500 μm and about 800 μm, or about 750 μm and about 800 μm. In other embodiments, the length is about 600 μm, about 750 μm, about 800 μm, about 900 μm, or about 1000 μm.

In some embodiments, the electrodes have a tiered arrangement and thus would not all be of the same length. In such embodiments, the length of the electrodes may range between about 400 μm and about 800 μm.

The base width of the electrodes may be at least less than about 50% of the length, less than about 25% of the length, less than about 20% of the length, less than about 15% of the length, less than about 10% of the length, or less than about 5% of the length. In some embodiments, the base width is at least about 100 μm but no more than about 400 μm. In other embodiments, the diameter is about 200 μm, or about 300 μm.

The diameter of the electrodes may be at least less than about 50% of the length, less than about 25% of the length, less than about 20% of the length, less than about 15% of the length, less than about 10% of the length, or less than about 5% of the length. In some embodiments, the diameter is between at least about 0.1 mm and no more than about 5 mm. In some embodiments, the diameter is between about 0.5 mm and about 1 mm.

It may be desirable for one microneedle to penetrate more deeply into the skin as compared to another microneedle. The two microneedles may therefore terminate at different distances from the skin surface, or at different distances from an electrode mounting portion. In some embodiments, the two microneedles are different lengths. In other embodiments, the microneedles are the same length, and a mounting portion is configured so as to axially displace one microneedle relative to the other. For example, the mounting portion may be multi-levelled with a first electrode extending from a first level and a second electrode extending from a second level.

The electrodes may be provided in various arrangements, and the number of electrodes provided on the mount will depend on available surface area. The mount may comprise up to about 100 electrodes. In some embodiments, the mount comprises between at least 2 electrodes and less than about 50. In other embodiments, the mount comprises between at least 2 electrodes and less than about 30. In yet other embodiments, the mount comprises between at least 2 electrodes and less than about 20. In yet other embodiments, the mount comprises between at least 2 electrodes and less than about 10.

Typically, the arrangement will be of a relatively low density, as this is likely to [179].facilitate breach of the stratum corneum by the electrodes and may avoid potential problems with skin penetration by high density arrangements. In some embodiments, the mount comprises at least about 4 electrodes/cm², at least about 8 electrodes/cm², or at least about 16 electrodes/cm².

The electrodes may be arranged in pairs, in groups, or as a matrix. A pair arrangement would comprise an even number of electrodes. A group arrangement may comprise between 1 and about 5 groups, with each group comprising between about 4 to about 8 electrodes. A matrix arrangement may comprise either an even or odd number of electrodes as such an arrangement may or may not have the same number of rows and/or columns. In some embodiments, the electrodes are arranged in matrix selected from the group consisting of 2×2, 2×3, 2×4, 2×5, 2×6, 3×2 3×3, 3×4, 3×5, 3×6, 4×2, 4×3, 4×4, 4×5, 4×6, 5×2, 5×3, 5×4, 5×5, 5×6, 6×2, 6×3, 6×4, 6×5, and 6×6. In any arrangement, the electrodes may be spaced less than about 5 mm, about 4 mm, about 3 mm, about 2 mm, about 1 mm, or about 0.5 mm, and more than about 0.1 mm, from each other. The space may be measured from the centre-to-centre point of each respective electrodes.

Like the mount, the electrodes can be made from any suitable material as described elsewhere herein.

Typically, the mount and electrodes are fabricated from dissimilar materials.

The structure of the mount and/or electrodes can be fabricated using any suitable technique. For example, in the case of silicon-based electrode structures, this could be performed using etching techniques. Polymer or plastic electrode structures, for example, could be fabricated using additive manufacturing such as 3D printing, or moulding including injection moulding. Solid polymeric electrodes are commonly fabricated from moulding methods such as injection moulding and micro-moulding. Generally, the injection moulding process involves heating a material to its melting temperature Tm and subsequently adjusting the processing temperature in order to inject the material into a mould at a given speed. Although injection moulding equipment can be expensive, it is compatible with large scale manufacturing. UV rapid prototyping is a technology which may also be used to fabricate hollow polymeric electrode structures. This technique uses computer models to 3D print polymeric builds by guiding light over a photocurable material for selective polymerisation. Once electro-polymerised, the electrode structure is subject to a cleaning step prior to curing. Electrode structures may also be fabricated using micro-Computer Numerical Control (CNC) micromachining methods. This fabrication method provides a flexibility in fabrication of the mount and shape, spacing, tip geometry, and material(s) selection (e.g., soft metals such as aluminium, 316L stainless steel and copper, machinable polymers such as polymethyl methacrylate, and ceramics) for the electrodes. CNC-based fabrication methods are also highly cost-effective, precisely reproducible (e.g., 1 μm precision), likely to have less residual material, and suitable for automated fabrication.

In the context of an EAB sensor, each working electrode is loaded with aptamers having specificity for a certain target analyte. Thus, where it is desired to detect four analytes, it is necessary to assemble at least four different working electrodes.

The working electrodes may have an associated at least one counter electrode and an associated at least one reference electrode. Each working electrode may have a dedicated counter electrode, however in some embodiments the counter electrode is shared amongst some or all of the assembled working electrodes. Each working electrode may have a dedicated reference electrode, however in some embodiments the reference electrode is shared amongst some or all of the assembled working electrodes.

An EAB sensor potentially useful in the context of the present invention may be of the potentiometric, amperometric or conductometric type. In a potentiometric sensor, a local equilibrium is established at the sensor interface, where either the electrode or membrane potential is measured, and information about a sample is derived from a potential difference between two electrodes. Amperometric sensors rely on a potential being applied between a reference and a working electrode, so as to cause the oxidation or reduction of a redox-active species; with the resultant current being measured. Conductometric sensors rely on the measurement of conductivity at a series of frequencies.

EAB sensors are typically of the amperometric type, with the aptamer (such as DNA, RNA or XNA) being bound to the working electrode. Gold is often used as the probe surface for the working electrode. The aptamer has an associated redox-active species which acts as a reporter. The redox reporter is often methylene blue. Upon target (e.g., drug) binding, the aptamer undergoes a conformational change, bringing the redox reporter more proximal to the working electrode surface. This increase in proximity increases electron transfer from the redox reporter to the electrode. The increase in speed of electron transfer contributes to a change in Faradaic current that is detected by a potentiostat.

Aptamers are small (usually from 20 to 60 nucleotides) single-stranded RNA, DNA or XNA oligonucleotides able to bind a target drug with high affinity and specificity. Aptamers may be considered as nucleotide analogues of antibodies, but aptamer production is an in vitro cell-free process that is significantly easier and cheaper than the production of antibodies by cell culture or in vivo methods.

Aptamers are usually selected from combinatorial library having a vast number (up to 10¹⁸) of different oligonucleotides. While RNA aptamers provide a significantly greater structural diversity compared to DNA aptamers, their application is complicated by stability issues in the presence of RNases, high temperature and unfavourable pH.

Selection of an aptamer that is selective for a given drug may be facilitated by a process known as SELEX (systematic evolution of ligands by exponential enrichment). The process may be considered as two alternating stages. In the first stage, the library oligonucleotides are amplified by a polymerase chain reaction (PCR) to the desired concentration. For the selection of RNA aptamers, the single-chained oligoribonucleotides are generated by in vitro transcription of double-stranded DNA with T7 RNA-polymerase. For DNA aptamers, a pool of single-stranded oligodeoxyribonucleotides is generated by strand separation of double-stranded PCR products. In the second stage, the products of amplification are incubated with target drug and oligonucleotides which bind the drug are used in the next SELEX round.

Separation of oligonucleotides with higher affinity for target drug and removal of unbound oligonucleotides are achieved through intense competition for binding sites. The selection pressure rises with every SELEX round. Maximum enrichment of the oligonucleotide pool with aptamers with the strongest affinity for the target molecule is usually achieved after 5 to 15 rounds.

EAB sensors are typically incorporated into a circuit having a reference electrode. The reference electrode is the site of a known chemical reaction that has a known redox potential. For example, a reference electrode based on the silver-silver chloride (Ag|AgCl) redox pair has a fixed and known potential forming the point against which the redox potential of the working electrode is measured. Also typically included in the circuit is a counter electrode which functions as a cathode or an anode to the working electrode. Because the applied voltage bias does not pass through the reference electrode (due to an impedance of the potentiostat), any potential generated is attributed to the working electrode. Current is measured as potential of the interrogating electrode versus the stable potential of the reference electrode. The difference in potential produces the current in the circuit thereby generating an output signal. The signal quantifies target binding depending on electron transfer that is ideally stoichiometrically proportional to target binding.

The present apparatus, when assembled, is particularly suitable for use as a wearable apparatus, allowing measurements to be performed whilst the subject is undergoing normal activities and/or over a prolonged period of time. The wearable apparatus may be a collar, a bracelet or other suitable jewellery piece, a watch, a garment, a strap, an adhesive, or a patch. A person skilled in the art would appreciate that means may be provided to assist adhering and/or securing the wearable apparatus, when in use, to a subject, e.g., micro-anchors, or the like.

The wearable apparatus may comprise a housing structure comprising one or more other components, such as electronics processing unit. The electronics processing unit is configured to be in direct or indirect electrical communication with at least one conductive element, and generally will include any one or more of a power source, a data processing unit, an analogue front-end, and a wireless transmitter.

The housing structure may be configured to encase, at least partially, the apparatus, where the electrodes (such as microneedles) are exposed from a plane of the housing structure. The electrodes may be protected by a protective cover, which may be removed to expose the protruding electrodes before use.

The apparatus may further comprise means for monitoring temperature or pH of the biological fluid where validity of an output is dependent thereon, or where adjustment to operation or output is possible.

The housing structure may be configured to encase and be coupled to the apparatus by any appropriate mechanism. For example, electromagnetic coupling, mechanical coupling, adhesive coupling, magnetic coupling, or the like. In some embodiments, the coupling mechanism enables the apparatus and the housing structure to be attached and detached, which would enable the housing structure and its other components to be re-usable, while the apparatus can be discarded and replaced with another apparatus as necessary.

The wearable apparatus may further comprise a computer program product executable as a software application, resident on a mobile communication device in communication with the electronics processing unit, wherein the computer program product is able to control one or more of (i) detection of electrochemical measurements conducted at the electrode-based platform, (ii) data analysis, (iii) data transmission, (iv) apparatus configuration, and (v) apparatus power management. Examples of suitable mobile communication devices include, but are not limited to, smartphones, smartwatches, tablets, smartglasses, laptops or other personal computers.

In some embodiments, the apparatus itself comprises a processor with program instructions configured to drive onboard functions such as voltammetry, and transmitting output to a remote device via a wireless module, such as a Bluetooth™ module.

The present invention will now be more fully described by reference to the following non-limiting embodiments and drawings.

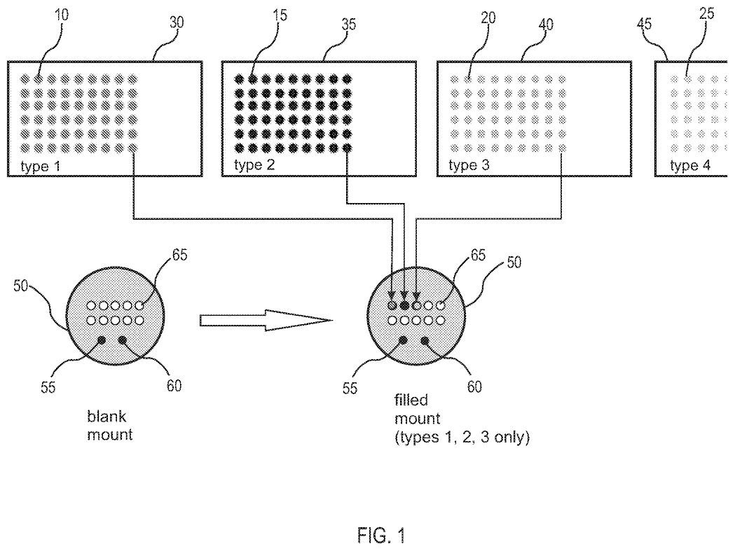

Reference is made to FIG. 1, showing an exemplary means for the selection and assembly of working electrodes in the production of an EAB sensor apparatus. In this example, a working electrode library has four electrode types 1, 2, 3 and 4 (one of each marked 10, 15, 20, 25 respectively). In practice, a significantly larger number of electrodes will be provided in the library. A plurality of each electrode type are grouped and retained in a dedicated holder (30, 35, 40, 45 respectively)

A blank mount (50) is provided, having already mounted thereon a counter electrode (55) and a reference electrode (60). The blank mount (50) further comprises a series of apertures (one marked 65) each of which is dimensioned to accept and retain one of the working electrodes (10, 15, 20, 25).

In the example of FIG. 1, it is desired to produce an EAB sensor apparatus capable of detecting three different analytes: the first analyte detected by the first electrode (10) type, the second analyte detected by the second (15) electrode type, and the third analyte detected by the third (25) electrode type. There is no need for the EAB sensor apparatus to detect any other analyte.

In the assembly method, working electrode of types 1, 2 and 3 (but not type 4) are removed from their respective holders and inserted into the apertures of the blank mount to produce the filled mount shown on the right.

Of course, in order to produce a functional sensing apparatus each of the working electrodes in the filled mount will be electrically connected to further hardware (not drawn) configured to provide electrical power, and to accept electrical signals from the electrodes. The hardware may have access to software instructions directing the delivery of electrical power (for example, with potential varying according to a square waveform) and analysing electrode output.

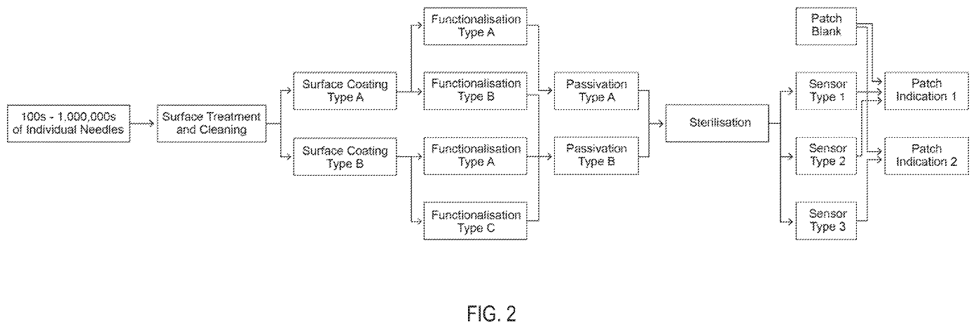

FIG. 2 shows a production method more broadly, with the steps of FIG. 1 being shown in the final two stages. The process of FIG. 2 commences with thousands of microneedles which will be treated by various alternative means at various steps to provide working electrodes of different types as shown in FIG. 1.

Staying with FIG. 2, each and every electrode is subjected to surface treatment and cleaning. One of two alternative surface coatings (A or B) may be applied (for example a certain metal coating). Each of those A or B coated electrodes can then be functionalised with analyte specific aptamer (type A, B, or C). A further passivation step (A or B) may then be applied, followed by a mandatory sterilisation step. The resultant electrodes (termed “sensor type” in the drawing) may be various mounted on a patch blank to provide a customised set of electrodes for use in analyte detection

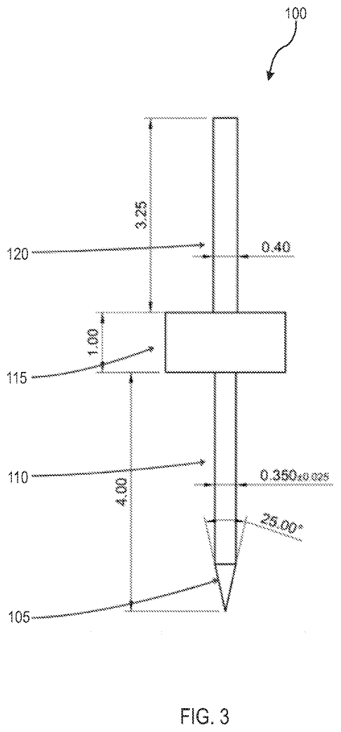

Turning now to FIG. 3 there is shown an exemplary solid microneedle electrode (100) useful as a working electrode in the context of the present invention. Where a working electrode is a microneedle, advantage is gained where a solid microneedle (as compared to a hollow microneedle) is used. Hollow microneedles are difficult to produce at scale given the need to drill or injection mould holes smaller than 0.3 mm, and the placements are difficult to achieve in terms of meeting a given tolerance. Moreover, the analyte detecting surface area is relatively low, given that it is a subset of the inner diameter of the hollow microneedle. In addition, if the detecting surface is recessed below the face of the hollow needle, changes in concentration will need to equilibrate through diffusion. Even small gaps may result in long lag times. Air may even become trapped in this location which could result in apparatus failure.

Staying with FIG. 3, the microneedle electrode (100) has a pointed terminus (105) configured to pierce the skin of a subject.

Superior to the terminus (105) is a detecting region (110) having analyte-specific aptamer coated thereon which functionalises the electrode to specifically detect a certain analyte.

Superior to the detecting region (110) is an expanded region (115) which functions to assist in handling, aligning the needle for correct insertion into a mount, and for abutting the mount once inserted.

Superior to the expanded region (115) is an electrical connection region (120) configured to make electrical connection with further hardware of the apparatus to allow for powering and accepting signal from the electrode via an interface. This interface is preferably of low electrical and mechanical resistance, easy to align, and tolerate some level of flex and compression. Conductive, compressible foams, plastics, silicones, or epoxies which may be independently placed on each needle, or if anisotropic (z-axis conductive) may be placed over all the assembled needles at once.

The dimensions (shown in mm) and angle are exemplary only and should not be considered limiting on the electrode drawn in FIG. 3.

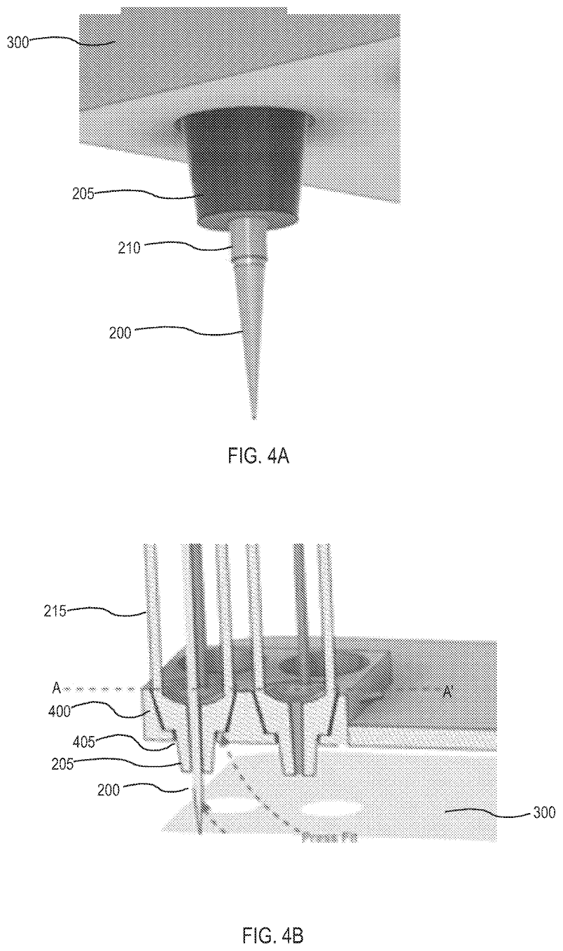

Reference is now made to FIG. 4A and FIG. 4B showing an embodiment of a functionalised microneedle (200) having an over-mould portion (205). The over-mould portion (205) is shown extending through the lower part of the enclosure (300). The microneedle has an electrical isolation coating (210) which extends upwardly into the over-mould (205).

Further detail of the embodiment of FIG. 4A and FIG. 4B. The over-mould portion (205) has support structures (one marked 215) which facilitate handling of the functionalised microneedle electrode (200) and insertion into the mount (400) via the aperture (405). The over-mould portion (205) and mount (400) engage by way of a press fit.

Once press fitted, the support structures (215) are removed by cutting at the level marked A-A′.

The press fit may on its own function as a watertight seal to prevent the entry of fluid (such as sample fluid, or an in situ biological fluid such as ISF) into the overlying electrical connections and electronics. Other or additional means for effecting a seal will be apparent to the skilled artisan having the benefit of the present specification and include flexible seals, curable materials, threaded connections, and the like.

In some embodiments all assembled electrodes have a consistent base material and/or geometry and/or dimension. This is facilitated by the large-scale batch-wise production of electrodes for subsequent functionalisation with aptamer as required. The metal composition and shape of the base electrode material can be modulated to meet several requirement including electrochemical surface area, surface roughness, retention in a mount through the inclusion of ramped faces or facets (barbs and the like), increased surface area through macroscopic geometry changes (ridges, eyes, grooves and the like), and surface area isolation through the inclusion of a dielectric masking layer.

With regard to electrochemical surface hardness, the electrode may be composed of an alloy to increase its hardness and reduce the risk of damage to the electrode. The surface of the electrode may be plated with gold, with the underlying electrode being preferably an alloy including cobalt, beryllium, or nickel.

Where the working electrode is a microneedle configured to pierce the skin and contact the ISF or other biological fluid, the shape and/or material may be optimised for that purpose. With regard to shape, the electrode may comprise bevels (single, or multiple), grinds (conical, undercuts), or hollows (some chemistries may benefit from the use of a hollow needle). With regard to material, the microneedle may be formed of a steel base layer, with a gold or silver plating/coating. In some embodiments, the needle is a solid material (including gold or silver). Moreover, the needle may be an alloy to increase stiffness or retain sharpness.

Detection of two or more target analytes may be improved where the EAB sensor apparatus comprises two or more analyte-specific working electrodes and a reference electrode, and each of the working electrodes is spaced substantially equidistantly from the reference electrode. In addition or alternatively, detection of two or more target analytes may be improved where the EAB sensor apparatus comprises two or more analyte-specific working electrodes and a counter electrode, and each of the working electrodes is spaced substantially equidistantly from the counter electrode. In such arrangements, and where the EAB sensor apparatus is operated under voltametric conditions, undesirable peak splitting and/or peak broadening is avoided. The result is a single peak output on the voltametric curve and/or a narrow peak, such output allowing for improved detection of binding of a target on an aptamer-coated working electrode.

In some applications, a non-equidistant arrangement may be preferred, and in which case a departure of at least about 1%, 5%, 10%, 20%, 30%, 40%, 50%, 60%, 70%, 80%, 90% or 100% from equidistance may be implemented. Such applications may arise where peak splitting or broadening does not present as a substantial problem, and where flexibility as to the spatial arrangement of electrodes is needed. For example, the apparatus may need to be shaped, dimensioned, or arranged in a certain way that does not allow for the use of equidistant electrodes.

The present assembled apparatus may be operable by way of voltammetry, including square wave voltammetry, cyclic voltammetry, chronoamperometry, chronopotentiometry, impedance spectroscopy, electrochemically implemented surface plasmon resonance, optical waveguide lightmode spectroscopy, ellipsometry, and quartz crystal microbalance, or a field-effect transistor-based method. Generally, the EAB sensor apparatus will be configured to be operable by way of voltammetry (and particularly square wave voltammetry), and in that regard will be configured to connect to a voltage source and particularly a controllable voltage source. Voltage is applied across at least the working electrode and the counter electrode when both are in contact with a biological fluid.

The microneedle may have other features to facilitate assembly. Such features include a scaffolding that can be removed prior to or after assembly to aid in handling, an increase in size that simplifies handling or placement of the needles, incorporation of flats, grooves, or keyways in the design of the needle to increase the accuracy of assembly, over-moulds, or insertion moulded plastics around the electrode needle.

In addition to the advantages described above, the present invention provides benefit to the process for manufacturing sensor apparatus. A large number of individual analyte specific working electrodes may be prepared in a batch number of at least about 10,000 or 50,000 or 100,000. Larger batches present technical advantage as production conditions do not need to be optimised time and time again.

Economic advantage is also provided on the basis of scale, and the need to perform QA/QC processes less often. The individual electrodes of a batch may be incorporated on an as needed basis into a sensor apparatus. In this way, a manufacturer can respond to consumer demand by providing desirable combinations of working electrodes, thereby preventing inventory accumulating for less desirable combinations.

Independent assembly of electronic and mechanical components. If the independent needles are inserted into the bottom of the assembled apparatus, this enables asynchronous construction of the apparatus and the electrodes. This enables the apparatus to be assembled, sterilised, and configured independent of the sensors which is preferable as the sensors should preferably have limited exposure to oxygen, and may require special sterilisation techniques. Further, a large stock of apparatuses may be constructed prior to final assembly and configuration in a separate facility, by a contract manufacturer, or in advance of final configuration with a selection of working electrodes.

As a further advantage the same mechanical and electrical components may be used interchangeably across multiple different sensor types thereby reducing development cost and time. Moreover, large single batches of mechanical and electrical componentry may be prepared given the ability to later customise sensor apparatus later in terms of the analyte detected.

The functionalised surface of a working electrode is sensitive to physical abrasion. Using a mount or dielectric masking helps enable mechanical articulation of the component without contacting the functionalised surface.

Further advantage may be found in relation to tolerances at high speeds and volumes. In electrode manufacture, maintaining highly accurate and precise tolerances (<+/−25 um) is challenging at high volumes and speeds. By allowing for some variance in component position through the use a larger electrical connection and a press fitting, the tolerance requirement is reduced relative to other configurations (i.e., hollow microneedles) to a value closer than +/−100 um.

The present methods include the assembly of the two or more electrodes with an apparatus for contacting the electrodes to the skin of a subject. The electrodes (which may be microneedles) may be mounted on a movable portion of the apparatus, or some other part of the apparatus. In some embodiments, the moveable portion of travels in a non-linear path.

The non-linear path may be of a limited length, and where the path is arcuate of a limited degree measure. By this arrangement, a major moving part of the apparatus requires only a limited range of motion in the vertical direction to bear on and insert the microneedles into the subject's skin. The limited range of motion allows for the housing of the apparatus to assume a relatively low profile when viewed in the lateral direction. Thus, the apparatus rises to a relatively small height over the skin and is therefore less obtrusive to the subject.

Moreover, the non-linear path of the movable portion allows for a simplified mechanism to be used. For example, the movable portion may move by way of a simple flexing or hinging mechanism. These mechanisms require a relatively small number of components, allowing for overall a smaller, lighter, simpler, more reliable, and less expensive apparatus to be developed.

Certain embodiments of the invention have further features, which either when taken alone or in combination with other features provide further advantage or a further useful alternative to the prior art. Such embodiments will be more fully described by reference to the non-limiting preferred embodiments described infra.

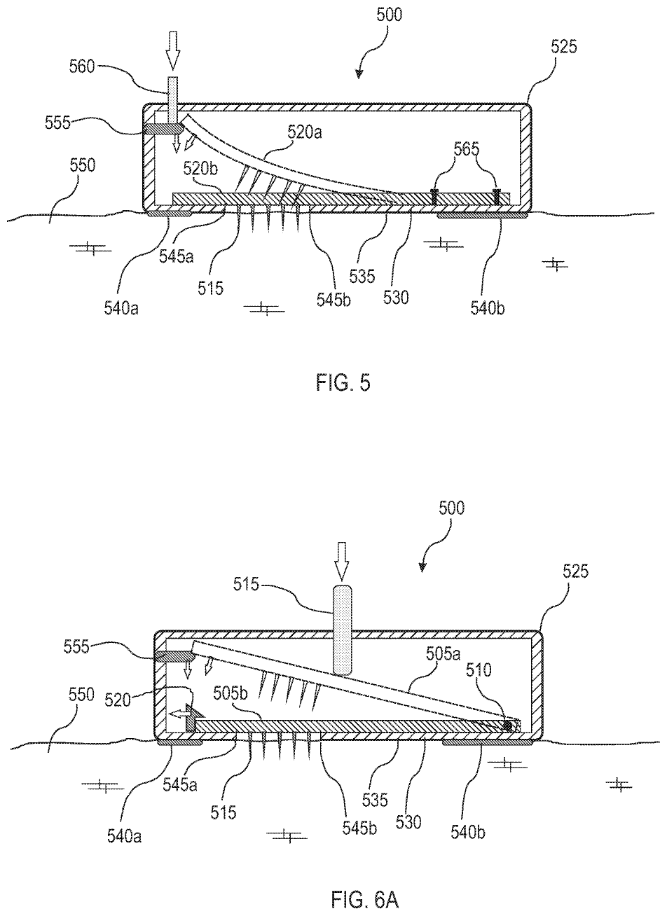

Reference is made to FIG. 5 showing a basic form of the present apparatus (500) having a microneedle array (one microneedle marked (515)) attached to movable portion, which in this embodiment is an elastically deformable arm (520). The arm (520) is biased to assume a linear configuration (520b), however is initially presented to the user with the arm flexed into an upward curvature as shown in the dashed representation (520a).

The apparatus (500) comprises a rigid housing (525) having a skin contacting portion (530) on its lower side which defines a downwardly facing skin contacting surface (535). The surface (535) is placed onto the subject's skin, and is retained therein by areas of a dermatologically acceptable adhesive (540a, 540b). A suitable adhesive will typically be capable of resisting water to allow the subject to bathe normally. The adhesive will typically have sufficient adhesion to inhibit detachment that may arise in the course of everyday activities such as dressing, undressing, sleeping, performing domestic chores, light to moderate intensity sporting activities, brushing past objects while walking and the like. The level of adhesive is typically not so great so as to cause any difficulty, unpleasant sensation, pain, irritation, or skin damage in removing the apparatus.

An exemplary adhesive is a synthetic rubber adhesive or tackified acrylic adhesive of the type used on medical tapes. A double-sided medical tape may be used, such as 3M™ 1577 tape, with one side adhering to the apparatus and the other adhering to the subject's skin.

The skin contacting portion (530) comprises a space (545), the margins of which are marked (545a) and (545b). The space (545) provides a passage through which the microneedles (515) pass, allowing the terminal regions of the microneedles to penetrate and embed into the underlying skin (550) when the arm (520) is in the linear position (520b).

The arm (520) is retained in its flexed state by the ledge (555) which functions as a releasing means. When the user wishes to insert the microneedles (515) into the skin (550) they depress the button (560) as shown by the arrow. The lower face of the button (560) bears on the ledge (550), and because the ledge (555) has some ability to deform (being fabricated from a rubber-like material, or formed from a flexible projection of the inner face of the housing (525) for example) it bends downwardly under the force so as to release the edge of arm (520a). The elastic nature of the arm (520a) causes it to rapidly return to its biased linear position (520b) thereby forcing the microneedles (515) into the underlying skin (550). The ledge (555) is configured so as to exhibit sufficient resilience to resist the biasing force in the arm (520a) however that resilience is not sufficient to resist the downward force exerted by the button (555) when depressed.

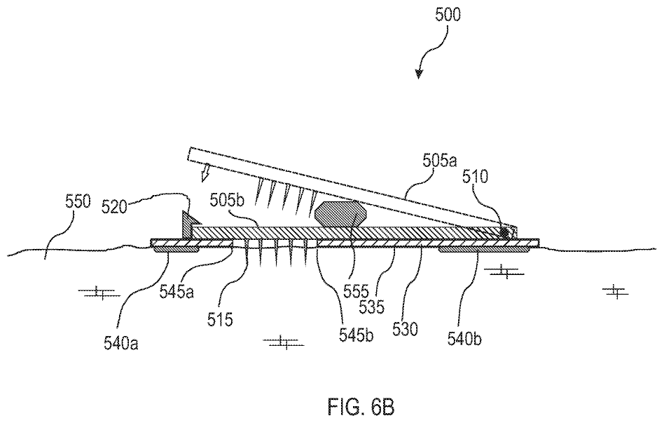

In the embodiment of FIG. 5, the arm (520) is fixed at one end to the housing (525) by the fasteners (565). While the arm (520) is flexible, the flexibility is not so high so as to easily move away from position (520b) when in place on the skin (550) of a subject. As will be appreciated, any movement of the arm (520) away from position (520b) may cause the microneedles (515) to withdraw from skin (550). Given the bias of the arm (520) toward the position (520b) there may be no need for a locking mechanism to maintain the arm in position (520b). However, if required a suitable locking mechanism is described infra for the embodiment of FIG. 6A.

FIG. 6A shows an alternative basic form of the apparatus (500) whereby the arm (505) is rigid and hinged to the housing (525) by way of hinge pin (510). The embodiment of FIG. 6A operates similarly to that of FIG. 5 so far as a ledge (555) acts as a releasing means. However, in the embodiment of FIG. 6A the button (515) acts on the rigid arm (505a). The rigid arm (505a) transfers the force of the button to the deformable ledge (555) causing the ledge (555) to bend and therefore release the free end of the arm (505a). The button (515) continues to be depressed by the user until the arm assumes the position (505b), and in which position the microneedles (515) are embedded into the skin (550). Again, a point on the free end of the arm (505a) travels along a non-linear path, and in this embodiment the path is an arc that is a segment of a circle, the origin of the circle being at the hinge pin (510).

It will be appreciated that the hinged arrangement of the FIG. 6A embodiment provides no resistance to the arm (505) hinging away from the position (505b) while the apparatus is being worn. A danger that the microneedles (515) withdraw from the skin (550) whilst in situ therefore presents. Accordingly, a locking mechanism is provided to maintain the arm in position (505b). The mechanism comprises a deformable latch (520), being fabricated from a material with some flexibility or from an internal projection formed from the housing (525) material, for example. The latch (520) has a sloped upper face, and upon contact with the rigid arm (505) the entire latch (520) is forced to bend to the left (as drawn) under the force being applied by the user via the button (515) and the sloped upper face. Once the terminus of the arm (505) clears the lower corner of the sloped upper face, the latch (520) resumes its normal upright position (as drawn) and the free end of the arm (505b) seats securely in the recess at the base of the latch (520).

An alternative to the embodiment of FIG. 6A is shown at FIG. 6B. In FIG. 6B, the apparatus (500) is devoid of an upper housing. The arm (505a) is maintained in position by the releasing means (555), which is removable by the user in this embodiment when the apparatus (500) is applied to a subject. After removal of the releasing means (555) the arm (505a) is depressed downwardly by the user so as to assume the second position (505b).

It will be noted in the embodiments of FIG. 5, FIG. 6A and FIG. 6B that when released from the ledge (555), the free end of the arm (520 or 505) moves in a non-linear manner as it returns to its biased position. If a single point on the free end of the arm (520 or 505) is considered, that point travels along a non-linear path which describes an arc. In the context of the present invention, the terms “arc”, “arcuate”, and similar terms refer to a curve joining any two points. The term “arc” should not be interpreted restrictively to mean only a segment of a circle, although in some embodiments it is a segment of a circle (see the embodiment of FIG. 6A for example).

As will be clear from the basic embodiments of both FIG. 5 and FIGS. 6A and 6B, in each case the arm (520 or 505) travels a relatively small distance when transitioning from the first position to the second position. Indeed, in these embodiments (and certain other embodiments) the apparatus is deliberately configured such that the arm is not able to travel along any path that is outside that between the first and second positions. Put another way, the apparatus may be configured such that the arm is unable to travel along any path that is outside the shortest distance between the first and second positions.

By placing limits on the path along which the arm may travel, advantage is provided in so far as the height of the apparatus (in the vertical direction, as drawn) is also limited. Accordingly, the apparatus may assume a low profile (in a dimensional sense) extending above the subject's skin a relatively short distance.

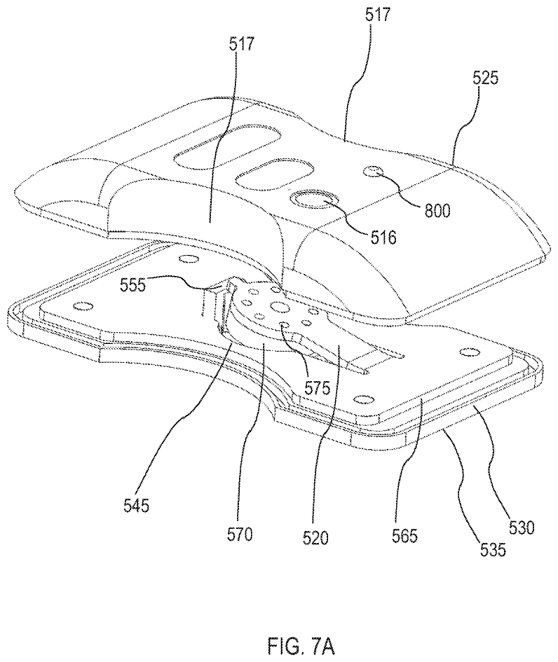

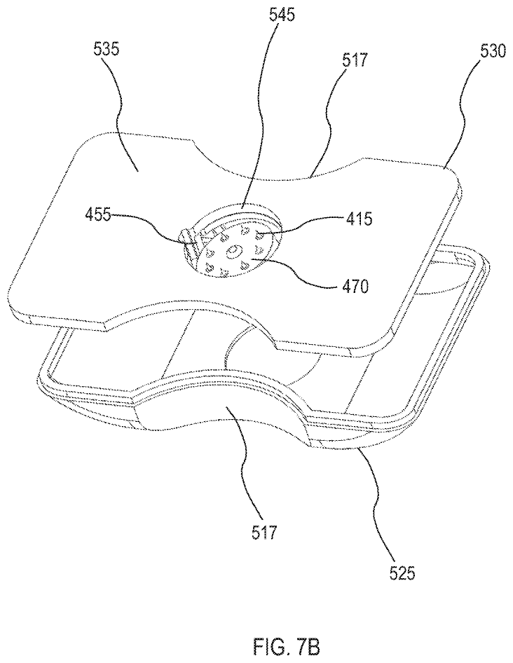

Turning now to FIG. 7A and FIG. 7B, there is shown a preferred apparatus constructed generally in accordance, and operable generally consistently with, the embodiment of FIG. 5. The arm (520) is formed integrally with a PCB (565) carrying the various electronic components required for operation of the apparatus. The PCB material is elastically deformable allowing the arm (with attached microneedles at the terminals) to flex upwardly as drawn to position the arm in the first position, but when released to assume the second position due to the natural bias in the arm toward the second position.

The arm (520) is maintained in the first position by the arm (520) terminus resting on the ledge (555) as shown most clearly in FIG. 5A. In this position, the microneedles (not shown, but extending downwards from 570) are retained within the apparatus with no part extending through the spaces (545). This is the configuration in which the apparatus is provided for use, and in which it is applied to the subject's skin.

The arm (520) has connected thereto a microneedle mounting block (570) supporting the microneedles. The mounting block (570) also contains conduits (not drawn) to carry electrical current from each of the microneedles (515) to one of a number of connection points (575) of the PCB (565). By this arrangement, electrical signals may be conveyed to and/or from microneedles embedded in the subject's skin. For example, the apparatus may be configured as a sensor with the microneedles configured to contact a biological fluid in the subject's body to detect an analyte therein. The biological fluid may be, but is not limited to, interstitial fluid, blood, or a mixture thereof. The electrical signals from the microneedles are conveyed to the PCB for amplification, filtering, encoding, analysis, transmission, or any other electrical or electronic process.

In this embodiment, the PCB serves the dual function of carrying the apparatus electronics and also as motive means for moving the microneedles from a position internal the apparatus to an external position. The PCB material has been found to be well suited to providing the limited range of motion preferred for the arm of the present apparatus. By this arrangement, the number of components in the apparatus is lessened.

The upper face of the housing (525) reveals the actuating surface of a button (516) which is depressible by the finger of a user. The button (516) is biased upwardly (as drawn) by a spring, or due to it being formed integrally with the housing (525) material. In the latter form of biasing, the button (516) may be mounted on an arm which is integral with the housing material and biased such that the upper surface of the button (516) is coplanar with the housing (525).

A lower portion (not visible) of the button (516) bears on the upper surface of the arm (520), the upper surface being the rear surface of the PCB (565) such that depression of the button (516) urges the arm (520) downwardly so as to release from the ledge (555) and assume the second position. In the second position, it will be appreciated that the microneedles will extend through respective spaces (545) and embed into the underlying skin (e.g., the epidermis, the dermis, or the hypodermis of the subject).

The natural bias of the PCB (565) material toward the second position is sufficiently strong for the arm (520) to remain in the second position without the need for any means of locking the arm in the second position. Accordingly, microneedles are able to remain embedded in the subject's skin for an extended period.

In an alternative embodiment, the arm (520) has a curved configuration when in the second position, and is naturally biased away from the second position. In another embodiment, the bias of the arm (520) toward the second position is not sufficiently strong so as to prevent any movement away from the second position. In such embodiments (and other embodiments) a locking mechanism may be provided to prevent movement of the arm away from the second position such that the microneedles do not retract into the apparatus and remain embedded in the skin. A suitable locking mechanism is the latch mechanism as disclosed in relation to other embodiments herein. Other locking mechanisms will be apparent to the skilled person having the benefit of the present specification.

The housing (525) comprises opposed depressions (517) to facilitate gripping between the user's thumb and second finger, and holding the apparatus against the skin's surface. The user's first finger is free to actuate the button (516) so as to embed the microneedles into the underlying skin.

The skin contacting surface (535) may have a dermatologically acceptable adhesive layer (not drawn) applied thereto so as to maintain the apparatus in situ on the subject's skin for an extended period. The adhesive layer can cover a portion or substantially all of the skin contacting surface (535). A manually releasable flexible layer may cover the adhesive until the apparatus is to be applied to the skin, as described for other embodiments of the apparatus as described herein.

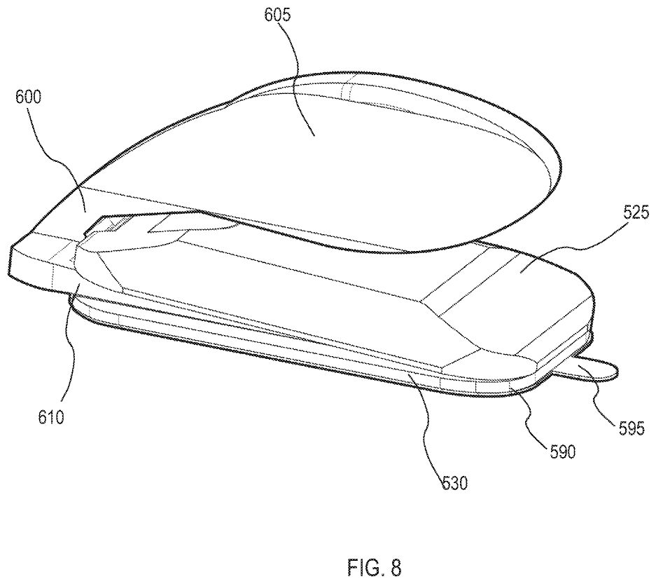

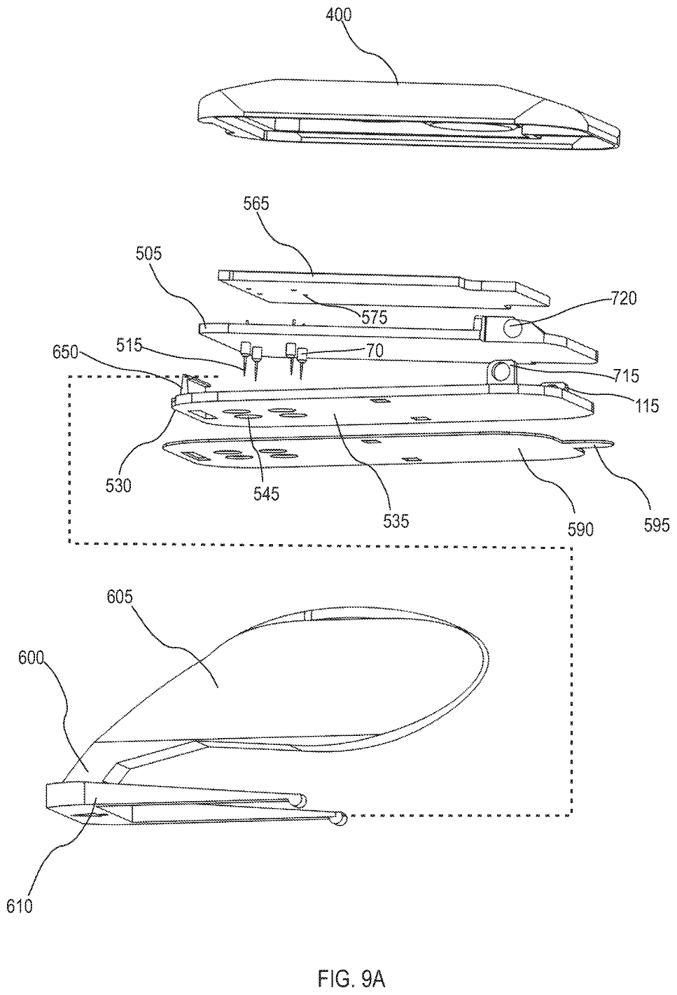

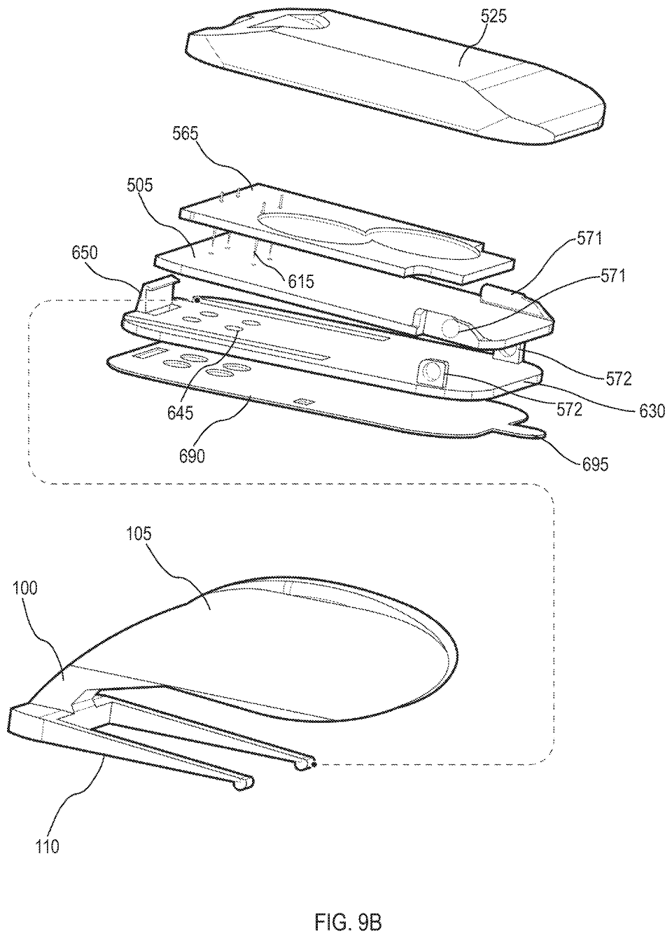

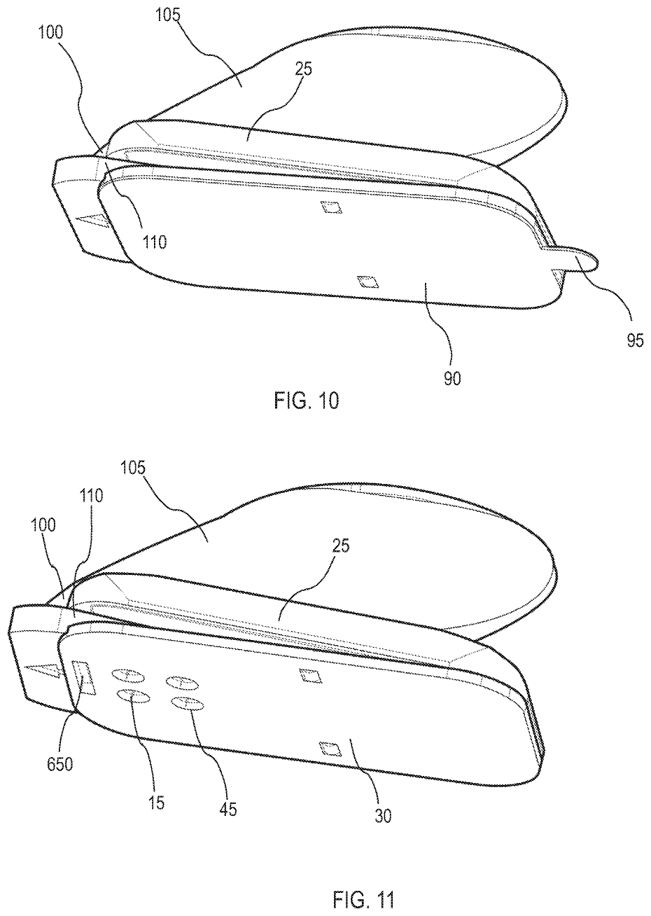

Turning now to FIG. 8, FIG. 9A, FIG. 9B, FIG. 10 and FIG. 11, there is shown a preferred apparatus constructed generally in accordance and operable generally consistently with the embodiment of FIG. 6B.

The embodiment comprises an upper housing portion (525) and a skin contacting portion (530). Also provided is a removable flexible layer (590) being graspable by way of the tab (595), the removal of which exposes a dermatologically acceptable adhesive on the skin contacting surface. As explained supra, the adhesive is for the purpose of retaining the apparatus on the subject's skin for an extended period. The flexible layer (590) functions to prevent curing or drying of the adhesive, prevent contamination of the adhesive layer before use and/or premature attachment of the adhesive to packaging, or to other surfaces. In a particularly preferred embodiment, in addition to covering the adhesive layer, the flexible layer (590) extends over the spaces (545) to prevent contamination of the microneedles and also help prevent unintended needle-stick injuries to a user.

The apparatus may have a retaining portion functioning to retain the apparatus on the skin such that the projecting portions remain in contact with a biological fluid of the subject. The retaining portion may be dedicated to that function, or may perform another function.

In many circumstances, a retaining portion being or comprising a dermatologically acceptable adhesive will be useful. Adhesives allow for simplicity in application of the apparatus by a user, often requiring only the removal of a protective backing sheet to expose the adhesive and then contacting the exposed adhesive to the skin. This method of application is similar to the application of a sticking plaster, and is therefore already a familiar process to users.

As an alternative to the use of adhesives, the retaining portion may be some mechanical means for maintaining the apparatus in the required position on the skin. For example, the apparatus may comprise a dedicated strap that engages about limb that is adjustable so as to keep the apparatus firmly applied to the subject. As an alternative, the apparatus may be incorporated into a wearable item such as a glove or a shirt, or an item of jewellery such as a ring which functions to retain the apparatus in position. The apparatus may be configured to engage with a discrete wearable item (such as by complimentary hook-and-loop means), or may have the wearable item integral therewith.

In some embodiments, the apparatus is retained simply by the wearable item bearing against the housing. For example, the retaining portion may be a snug-fitting elasticised glove which is worn over the apparatus.

In some embodiments, the retaining portion is any surface or part of the apparatus which contacts the skin of the subject, with a feature of the subject being at least partially responsible for maintaining the apparatus in place on the subject. For example, the apparatus may be configured to be retained between two parts of the body normally in close apposition, or within an existing anatomical structure. The apparatus may be shaped and/or dimensioned to be retained between the toes, the buttocks, in the groin, in the buccal cavity, in a nostril, in the ear canal, or in the umbilicus.

In other embodiments the apparatus housing is shaped and/or dimensioned to snugly fit over a digit, a toe, or an ear, for example. The apparatus housing may be elastically deformable, composed of a rubberised material for example, and configured to be stretched over any anatomical part (such as a finger)

Each of the aforementioned embodiments is considered to be a retaining portion in the context of the present invention.

The apparatus further comprises a releasing member (600) having a grasping portion (605) and a wedging portion (610), the function of which will be more fully described infra.

Turning now to the exploded views of FIG. 9A and FIG. 9B, components that are analogous to those in earlier figures will be immediately apparent.

In this embodiment, the motive force responsible for moving the arm (505) thereby urging the microneedles (515) into the underlying skin is provided by the user. In use, the user places a finger on the upper housing (525) and pushes downwardly. Furthermore, the arm (505) is movable by way of a hinging arrangement.

The hinging arrangement is provided by way of opposing lugs (715) extending from skin contacting portion (530), each lug comprising an aperture. The arm (505) comprises opposing laterally extending discs (571), each of which seats into an aperture of the lugs (572). It will be apparent that the arm (505) is able to hinge relative to skin contacting portion (530) to allow movement from the first position to the second position.

The arm (505) is presented to the user having the arm in the first position. The arm (505) is maintained in the first position by the wedging portion (610) of the releasing member (600). Before removal of the releasing member (600) the wedging portion (610) inserts between the skin contacting portion (530) and the arm (505), thereby keeping the microneedles within the apparatus.

When intending to apply the apparatus to the subject's skin, the user removes the flexible layer (590) by pulling on the tab (595) to expose the adhesive layer on the skin contacting surface (535). The apparatus is then applied to the skin, with the adhesive maintaining it in situ for an extended period.

Once the apparatus has been applied to the skin, the user grasps the grasping portion (605) and pulls laterally to the left (as drawn), so as to completely remove the releasing member (600). The releasing member (600) has no further function and is discarded at this juncture. By removal of the releasing member (600) the arm (605) is released from the first position and permitted to move (under a downward force exerted by the user) into the second position whereby the lower face of arm (605) contacts the upper face of the skin contacting portion (530). In the second position, the microneedles (515) extend through the spaces (545) and into the underlying skin.

As will be appreciated, the releasing member (600) may be configured to prevent the upper housing (525) of the apparatus from closing to the skin contacting portion (530) when not intended by the user. The releasing member (600) is inserted or otherwise juxtaposed between the upper housing (525) and the skin contacting portion (530) to prevent closure of the upper housing (525) towards the skin contacting portion (530) sufficient to allow the tips of the microneedles (i.e., projecting portions) to protrude from the base of the holes in the skin contacting portion (530). Preventing closure also prevents movement of the arm (505) from the first position to the second position. Thus, when the releasing member (600) is in place, the tips of the microneedles cannot be inadvertently accessed to cause microneedle contamination or injury. In using the apparatus, the user removes the releasing member (600) as a step in the use process. In a preferred embodiment of apparatus use, the user first adheres the apparatus to the subject's skin and then removes the releasing member (600), prior to pressing the upper housing (525) to insert the microneedles into the skin.

Prior to removal by the user, the releasing member (600) can be kept in place by any one of a variety of features. In one example the releasing member (600) comprises protrusions that fit into recesses in either the upper housing (525), the skin contacting portion (530) or both the upper housing (525) and the skin contacting portion (530) to assist in retaining it in place until intentionally removed. In another example the releasing member (600) is designed to be slidably assembled to the skin contacting portion (530) or upper housing (525), such that friction between the releasing member (600) and either the upper housing (525) or the skin contacting portion (530) assists in keeping it in place until intentionally removed. In yet another example magnetic force may be used to assist in keeping the releasing member (600) in place. In one embodiment of the invention, a magnet mounted within the releasing member (600) is positioned so as to be proximal to a Hall effect sensor positioned in either the upper housing (525) or the skin contacting portion (530), when the releasing member (600) is in place. According to this embodiment, when the releasing member (600) is removed by the user, the Hall effect sensor detects the removal of the magnet and causes the apparatus to take some action, such as powering up the electronic circuitry ready for use, converting it from sleep mode to active mode. It is to be understood that the above are examples of possible methods for assisting in retaining the releasing member (600) in place prior to intentional removal that may be used alone or in combination and that other methods as known in the art can also be used alone or in combination with the examples given.

In some embodiments of the invention, the releasing member (600) can also function as a covering element that is used to cover the microneedles after the apparatus has been removed from the subject. In a preferred example of this embodiment the locking element is located on the upper housing (525), extending down towards the skin contacting portion (530). The releasing member (600) comprises a groove that allows the releasing member (600) to slide past the locking element when the releasing member (600) is being withdrawn from the apparatus, while keeping the face of the releasing member (600) facing the upper surface of the skin contacting portion (530) continuous. In use, a releasing member (600) according to this preferred embodiment is removed by the user prior to pressing the upper housing (525) to insert the microneedles into the subject's skin and retained by the user. After the apparatus is removed from the subject post use, the user is instructed to adhere the releasing member (600) to the adhesive layer on the lower surface of the skin contacting portion (630) to cover the protruding microneedles. In another example of this embodiment, the releasing member (600) is flexibly attached to the apparatus such that the releasing member (600) can remain attached to the apparatus after it has been withdrawn by the user and then repositioned to cover the protruding microneedles after the apparatus has been removed from the subject post use. In yet another example of this embodiment, the releasing member (600) and the upper housing (525) are designed such that the releasing member (600) can be slidably or otherwise engaged with the upper housing (525) once it has been removed, where it is intended that the releasing member (600) be stored while the apparatus is in use and removed to be used as a covering element after the apparatus has been removed from the subject.

In some embodiments, of the apparatus is configured to facilitate the user in removing the apparatus from the subject. As will be appreciated, the use of an adhesive layer may result in difficulty in removal of the apparatus from the skin. Examples of such configuration include leaving a portion of the skin contacting surface (535) uncoated with adhesive, such that a gap is present between the subject's skin and the surface (535), wherein the user uses the gap as a leverage point to assist in pulling the apparatus away from the skin by breaking the adhesive bond. In another example, a leverage mechanism not located on the skin contacting surface is incorporated to allow a taller gap than that created by the absence of adhesive on a portion of the skin contacting surface. In yet another example, a tab extending beyond at least one edge of the skin contacting portion (30) and attached to the adhesive layer can be incorporated, where the user pulls on the tab with sufficient force to cause the adhesive layer to stretch and yield, further causing the adhesive to delaminate from the skin contacting surface (535) and the skin.

In some embodiments of the invention, the apparatus is designed such that the releasing member (600) is locked into place in its position prior to apparatus use unless pressure is applied to the upper housing (525). This embodiment is intended to further ameliorate the risk of the releasing member (600) being prematurely withdrawn. In an example of this embodiment, there are features on the releasing member (600) and on at least one of the upper housing (525) and skin contacting portion (530) that are lockably engaged when the upper housing (525) is not being pressed. When the upper housing (525) is depressed, the feature on at least one of the upper housing (525) and skin contacting portion (530) is distorted, so as to disengage the releasing member (600) and allow it to be withdrawn.

In yet other embodiments, the releasing member (600) need not be removed from the apparatus by the user. According to these embodiments, the releasing member (600) comprises a flexible element of sufficiently high stiffness that it does not substantially deflect when subjected to closing forces likely to be present on the apparatus during manufacture, storage and in the user's hands prior to application to the subject, but flexible enough that it deflects when the user intentionally applies a closing force to the apparatus when it is applied to the subject's skin. In so flexing, the releasing member (600) is deflected, allowing the upper housing (525) to close towards the skin contacting portion (530). In these embodiments, the releasing member (600) could also function as the locking element, or the releasing member (600) could be separate from a locking portion. In some of these embodiments, a structure such as that labelled as (650) in FIG. 9A, FIG. 9B, and FIG. 11, forms the releasing member (600).

Each space (545) of the apparatus is dimensioned such that a microneedle can extend through it clearly, with at least a tapered part of the microneedle not impacting the sides of the hole during insertion. In some embodiments the holes may be of sufficient cross-section such that no part of the microneedle will contact the sides of the space during insertion. In other embodiments, at least a part of the hole along its length will have a cross-section such that a portion of the length of the microneedle contacts the sides of the hole during insertion. According to this embodiment the hole functions to help support a portion of the length of the microneedle to assist in preventing bending of the microneedle as it is inserted.

In some embodiments of the apparatus, the skin contacting portion (530) comprises further spaces or depressions configured to accept protrusions on the releasing member, to assist in retaining the releasing member until it is removed by the user. In addition, or alternatively, the skin contacting portion (530) comprises protrusions designed to be accepted into recesses in the releasing member to assist in retaining the releasing member in place until deliberate removal by the user.

The embodiment depicted in FIG. 8, FIG. 9A, FIG. 9B, FIG. 10, and FIG. 11, comprises a locking portion in the form of a latch (650) which permanently locks the arm (505) in the second position preventing the arm (505) from any hinging movement. In the drawn embodiment, the latch (650) is a simple unitary member capable of deflecting in response to movement of the arm (505) toward the closed position, but then returning to its original position when the arm (505) is in the second position (505b), thereby locking the arm (505) in place.

Rather than act on the arm (505), a locking portion may act on another component of the apparatus, that component in turn locking the arm in place. For example, the locking portion may act on the upper housing (525), with the upper housing (525) in turn retaining the arm (505) in the second position. In a further alternative the locking portion may act on the PCB (565), with the PCB (565) in turn retaining the arm (505) in the second position.

In other embodiments, the locking portion comprises a recess into which a protrusion on the upper housing (525) is inserted to lock the upper housing (525) in a closed position (i.e., with the arm (505) in the second position). In one embodiment, the locking portion comprises a flexible element that is designed to allow the locking portion to move when impinged upon by the upper housing (525), so at to allow the housing (525) to close relative to the skin contacting portion (530) and whereby once the upper housing (525) has closed, allows the locking portion to move to lock in place the upper housing (525) in the closed position. In one embodiment, the apparatus comprises a protrusion on the upper housing (525), designed to be inserted into a recess in the locking portion, the protrusion comprising a flexible element to allow the protrusion to move, allowing the upper housing (525) to close relative to the skin contacting portion (530) and whereafter the housing (525) has closed relative to the skin contacting portion (530) the protrusion moves to be inserted in the recess in the locking portion, so as to lock the upper housing (525) in the closed position. The flexible element may comprise a shaft that is sufficiently deformable to allow the upper housing (525) to close without yielding of the shaft, so that the flexible element will try to return to its original position post the upper housing (525) closing. In a less preferred, but nonetheless functional embodiment, the flexible element comprises a coil spring.

A flexible element of the locking portion may be fabricated from any suitable material having the necessary stiffness and yield point. Examples of suitable material include non-crystalline plastics, crystalline plastics, sprung steel, unsprung steel, stainless steel, or other materials as are known if the art with suitable mechanical properties.

In a preferred embodiment of the invention, the locking portion is fabricated from the same material as the skin contacting portion (530), to facilitate the fabrication of a skin contacting portion with an integral locking portion.

In a particularly preferred embodiment of the invention, the force required to deflect or otherwise move the flexible element is designed to be large enough that the pressure the user needs to supply to deform the flexible element and thus cause the upper housing (525) to close towards the skin contacting portion, is sufficient to insert the microneedles into the skin. According to this embodiment, the flexible element of the locking portion is used to set the force necessary to close the apparatus (thereby causing the arm to assume the second position) and ensure that the force is sufficient to insert the microneedles in their intended position embedded in the skin.

In other embodiments, the locking portion comprises at least one adhesive region located on at least one of the lower surface of the upper housing (525) and the upper surface of the skin contacting surface (535). When the apparatus is closed, the one or more adhesive regions adhere the upper housing (525) to the skin contacting portion (530), locking the apparatus in the closed position.

In another embodiment of the invention, the locking portion can assume three different stable states. In a first state, the locking portion is in a disengaged configuration, before the upper housing (525) is pushed downwardly towards the skin contacting portion (530) to close the apparatus. In a second state, the locking portion is in a first engaged position. When the locking portion is in the first engaged position it serves to lock the microneedles (515) in the embedded position in the skin (i.e., the arm (505) being in the second position). In a third state, the locking portion is in a second engaged position. In this state, the locking portion locks the apparatus in the open position (i.e., with the arm (505) in the first position) with the microneedles withdrawn into the apparatus to ameliorate the possibility of needle-stick injury resulting from microneedles protruding after apparatus use. In an example of this embodiment, the locking portion comprises a user engagement portion, that can be gripped or otherwise engaged by the user, for example by engaging a fingernail under an overhanging ledge, so that the user can deflect the flexible portion of the locking portion. According to this example, to close the apparatus the user presses on the upper housing (525) and locks it in place, as in other embodiments disclosed herein. When it is desired to remove the apparatus from the subject, the user engages with the locking portion and deflects it in a first direction, so as to unlock the upper housing (525) from the skin contacting portion (525), and then deflect the locking portion in a second direction, to lock the apparatus in the open position (i.e., with the arm in the first position) with the microneedles in the withdrawn position. In a preferred embodiment of this example, in the first direction, the locking portion is moved is away from the body of the apparatus, and in the second direction, is towards the body of the apparatus. When deflected sufficiently in the second direction, the locking portion is designed, for example, to be stably engaged in a recess so as to prevent closure of the apparatus without intentionally doing so.

In some embodiments of the invention, a downward force on the microneedles when inserted into the skin is provided via the flexible element of the locking portion applying a downward force when the apparatus is locked in the closed position (i.e., with the movable arm in the second position). In some embodiments, effective locking of the movable arm in the second position is provided by a dedicated spring or other suitable biasing means. In other embodiments, the spiring or other biasing means is not dedicated to a locking function and may, for example, act also as a motive force in the movement of the arm from the first position to the second position. For example, a torsion spring may apply a closing torque at a pivot point (where present). In yet another example a flat, disk or coil spring is mounted to the rear of microneedles, such that when the apparatus is closed the spring is distorted or compressed so as to apply a downward force on the microneedles when the apparatus is in the closed position.

Although not an essential feature of the invention, the PCB (565) will be required for many applications where the microneedles are for the purpose of conducting electrical current to, from or through the skin. In that regard, the PCB may carry a microprocessor, and/or volatile electronic memory (such as RAM) and/or non-volatile electronic memory (such as ROM) and/or a wireless networking module (such as a Bluetooth™ module). The apparatus will of course comprise a power source, typically by way of a button battery.

The embodiment depicted in FIG. 7A further comprises a light emitting diode (LED) (800) viewable by the user. One function of the LED (800) may be to confirm to the user and/or the subject that the microneedles are properly embedded in the skin at application, and remain so for the extended period of wear

The LED makes electrical connection with the PCB (565), which in turn makes electrical connection with the microneedles (515). Proper embedment of the microneedles can be determined by reference to any one of more of current flow, resistance to current, or impedance between two microneedles.

Alternatively, proper embedment of a single microneedle can be determined by reference to any one of more of current flow, resistance to current, or impedance between the single microneedle and some other electrical contact of the apparatus with the skin. As an example, an electrically conductive pad can be placed against the surface of the skin, where in some examples the conductive pad is placed on the face of the housing that contacts the skin. This electrically conductive pad in concert with at least one of the microneedles complete an electrical circuit when the microneedle is inserted into the skin. Completion of this circuit is used to indicate correction insertion of the microneedles.

The electronics involved may be simple, any example of which being a biological fluid of the skin, such as interstitial fluid (which is naturally conductive) acts to complete a circuit including the LED. The assumption is made that proper embedment is indicated by the simple contact of a microneedle with a biological fluid. The LED is illuminated where the microneedle contacts the biological fluid (or vice versa) thereby providing a visual indication of correct embedment.

More sophisticated electronic arrangements may be required to provide a higher level of assurance of correct microneedle embedment. For example, regard may be had to whether or not a minimum length of microneedle is embedded, thereby providing an assurance of insertion of the microneedle to a certain minimum depth. The apparatus may comprise electronic means of measuring the quantum of a parameter such as current flow, with a higher current flow being indicative or more complete embedment of a microneedle. Program instructions executed by a processor on-board or otherwise associated with the apparatus may use as input a parameter such as current flow (possibly in conjunction with other physiological or environmental parameters) to provide an indication of the degree of embedment of the microneedle.

A further function of the LED may be to provide other information such as battery charge level. For example, the LED may be connected to a microprocessor capable of monitoring battery voltage, with the microprocessor causing the LED to blink red when voltage falls below a predetermined threshold value. That value may be a voltage that is somewhat above the minimum operating voltage to allow the subject time to access a replacement battery (or replacement apparatus where the battery is not user-serviceable) before the apparatus becomes inoperable.

In other embodiments, the LED may produce an output indication of a data connection status. For example, the LED may blink alternating red and green light to warn of a disruption in a wireless data connection with a remote device such as a smartphone. A smartphone may be responsible for processing sensor output, and warning the subject by an audible output when a threshold (such as glucose concentration) is breached. In such embodiments, the LED and an apparatus networking module may be connected to a microprocessor, the microprocessor monitoring the connection status of the module and causing the LED to produce an output when the connection is made and/or lost. While application software on the smartphone may be configured to alert the subject to a loss of data connection, the smartphone may lose power (by running out of charge, for example) and in which case the only means by which the subject could be alerted is by way of the apparatus itself.

Similar output functions to the LED may be provided by a buzzer or a miniature speaker to provide audio output comprehensible by the subject. The output may a tone, a series of tones, or a synthesised voice for example.

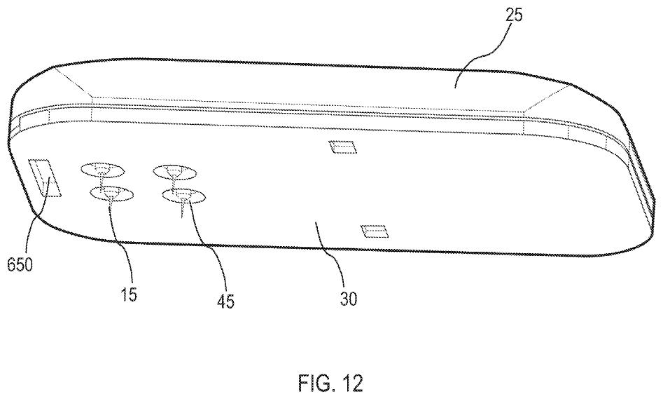

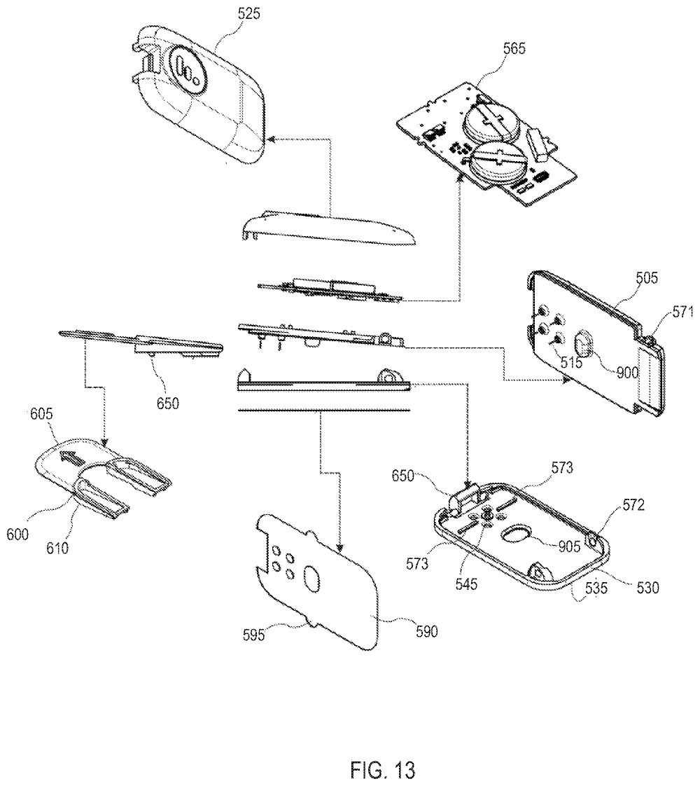

Reference is now made to an alternative embodiment of the apparatus shown at FIG. 13, being a modified version of the embodiment depicted in FIG. 8 through to FIG. 12. The embodiment of FIG. 13 includes a temperature sensor (900) which in operation extends through the space (905) in the skin contacting portion (530) so as to contact the surface of the subject's skin. The temperature sensor (900) may be a thermocouple or a thermistor, for example, in operable connection with a microprocessor on the PCB (565). The temperature sensor may directly contact the skin, or may be separated from the skin by way of a thermally conductive material.

The temperature sensor may be disposed within a pocket or other formation dimensioned to receive the temperature sensor. The pocket may be fabricated from a thin sheet-like material of a plastic, such as a thermally conductive plastic having a metal or other filler to facilitate transmittance of thermal energy from the underlying skin to the temperature sensor. The temperature sensor may be surrounded by a thermally conductive paste to facilitate transfer of thermal energy from the pocket wall to the temperature sensor.

The floor of the pocket may extend outwardly from the apparatus such that the floor of the pocket is pushed gently onto the skin surface when the apparatus is applied thereto, thereby facilitating transfer of thermal energy from the skin to the temperature sensor. It will be understood that overly firm pushing of the pocket floor onto the skin surface may force blood out the skin capillaries thereby artificially cooling the skin surface.

Preferably, only the floor of the pocket is fabricated from a thermally conductive material, with the remainder being fabricated from a material of low thermal conductivity. By that arrangement, thermal energy from the skin will not be routed away from the temperature sensor.

An insulating material may form a ceiling of the pocket to ensure thermal energy is retained about the temperature sensor and not lost to the internal cavity of the housing.

The pocket may comprise a space extending through the floor so that the temperature sensor can directly contact the skin surface. A temperature that is closer to the actual skin temperature would be expected given that thermal energy is not required to traverse any intervening material.

In a further modification, the temperature sensor may be an infrared sensor module, and in which case the material of at least the pocket floor should not substantively interfere with its operation. It is contemplated that a space could be formed in the floor to allow the infrared sensor module direct exposure to the skin surface to effect an accurate reading of skin temperature.

Signal output from the temperature sensor (300) may be used in calculations made by the microprocessor (or a remote microprocessor) to more accurately determine the concentration of a target analyte. For example, the microprocessor may have access to a range of stored calibrations curves, each curve having been performed at a given temperature. Based on the output of the temperature sensor (300), the appropriate calibration curve may be selected and a more accurate analyte concentration therefore determined.

The embodiment of FIG. 13 comprises a releasing member (600) having paired protrusions (a first protrusion marked (650), the second of the paired protrusions being obscured by the first). The protrusions (650) extend downwardly and through the spaces (573) in the skin contacting portion (530). The function of the protrusions (650) is to prevent lateral movement of the releasing member (600) until the lower face of the skin contacting portion (30) is pressed against the skin. The act of pressing against the skin causes the protrusions (650) to vertically exit the spaces (573) so as to allow the releasing member (600) to be pulled laterally away by the subject. This mechanism prevents the releasing member (600) from being inadvertently removed before the apparatus is properly applied to the skin surface. Absent such a mechanism, the microneedles (515) may be caused to prematurely extend through the spaces (545) and may become contaminated by contact with the air or an object, or become physically damaged by catching on clothing for example.

Some embodiments of the apparatus may require the upper regions of the microneedle to be electrically insulated to avoid the moist surface of the skin (as distinct from a biological fluid thereunder) forming a conducting path between microneedles.

As another means of controlling moisture, an absorptive material may be positioned on a microneedle mounting portion and proximal to the microneedle tips. In embodiments of the apparatus for sensing applications, the material is configured to absorb any excess fluid that may be produced by insertion of the microneedles in the skin to improve subject experience and to ameliorate any issues fluid contact with other parts of the apparatus, such as the electronic circuitry or electrical contacts, may cause. In embodiments of the apparatus such as fluid extraction applications, the material acts as a wicking agent to transport the fluid from the microneedle site to the required final site on the apparatus or external to the apparatus. In some embodiments the absorptive material is in the form of a sheet. In embodiments where it is desirable to prevent contamination or damage to the microneedles prior to insertion, the sheet comprises holes through which the microneedles pass, wherein the holes are dimensioned to be sufficiently large to prevent the absorptive material coming into contact with the microneedles during the microneedle insertion process, but sufficiently small to allow excess fluid exuding from the access penetration point created by a microneedle to contact and be absorbed by the material. In other embodiments, such as when the apparatus is intended to be used for fluid extraction, there are either no holes in the sheet of absorptive material, or the holes are dimensioned so that the absorptive material contacts the microneedle during and post insertion to aid in its wicking action. In the embodiment with no holes in the sheet the microneedles create holes when they pass through the sheet as part of the insertion process.

The present apparatus may be configured for use and/or used in any suitable application where microneedles are required to be embedded in a subject's skin for an extended time period.

Such applications include electrochemical aptamer-based sensing whereby a target analyte in a biological fluid is detected by binding to a capture entity such as an aptamer comprising a redox reporter. The capture entity may be covalently or non-covalently bound to the microneedles, with the redox reporter causing an electrical signal to be conveyed by the microneedles upon binding of the target analyte. The target analyte may be a drug or other exogenous species, or an endogenous species such as a hormone or a metabolite.

Where the microneedles function as electrodes to detect analytes present in the layers of the skin, the apparatus may comprise circuitry and components to excite the electrodes electrically and to receive, measure and process the electrical signals that result from the electrical excitation. According to this embodiment the microneedles may comprise a tip, a shaft, and a base, where electrical signals are generated at electrodes either coated on to the surface of or integral to the microneedle, transmitted along the shaft of the microneedle to the base of the microneedle, where electrical connection is made to the base or shaft of the microneedle to transmit the electrical signals to and from the electrodes to the electronic circuitry. The electrodes can be formed proximal to the tip of the microneedle, on at least a portion of the shaft of the microneedle and not proximal to the tip of the microneedle or both proximal to the tip of the microneedle and on at least a portion of the shaft of the microneedle.

The microneedles may be connected to the electronic circuitry by a variety of methods as are known in the art, for example, soldering, wire wrapping or sprung loaded pins. In one embodiment, the microneedles are mounted so as to pass through a plate or a block of dielectric material with the connection portion of the microneedles positioned at or above the surface of the plate or block distal to the microneedle tips. A zebra strip connection may be used to connect the microneedles to the electronic circuitry to facilitate robust connections without the need to precisely align the zebra connector with the microneedle ends, as least in one dimension.

Another potentially useful application is delivery of an active substance into the skin. The substance may remain predominantly in the skin, or may enter the systemic circulation. In such applications, the microneedles may be hollow, with the substance being delivered through needle lumens. Alternatively, the microneedles may be coated with the active substance, such that the substance is released instantaneously into the biological fluid or released gradually over an extended time frame. As a further alternative, the microneedles themselves may be dissolvable in the biological fluid and include the active substance within their bulk, such that active substance is released as the microneedles dissolve. The active substance may be a pharmaceutical composition (a small molecule, protein, peptide, or nucleic acid, for example), or an immunologically active composition (a collection of proteins, for example) for use as a vaccine.

A further potential application is for the delivery of electrical current to the skin for the purpose of muscle stimulation, or for the stimulation or inhibitions of a biological process of the subject. Similarly, the present apparatus may be used to detect electrical currents in the subject's skin, for example to detect nerve conductance.

In any of the above applications, the microneedles may be solid or hollow, as required or as desired.

Microneedle length may be selected according to a particular application. Typically, the microneedles will be required to extend at least below the stratum corneum. The depth of the stratum corneum varies according to location, that layer being relatively thick on the soles of the feet and relatively thin on the backs of the hands, for example. Accordingly, the length of microneedle extending beyond the housing may be adjusted according to the intended site of application.

In some cases, the microneedles may be required to extend well below the stratum corneum, and into the lower layers of the epidermis, the dermis and even the hypodermis, including the subcutaneous tissue. Again, the length of the microneedles extending beyond the apparatus may be set accordingly.

The skilled person will also appreciate the possible need to set microneedle length according to the intended subject. For example, relatively short microneedles will generally be required to effect contact with the subcutaneous tissue of a neonate subject, while for the same site an adult subject will require longer microneedles

It may be desirable in some applications for one microneedle to penetrate more deeply into the skin as compared to another microneedle. The two microneedles may therefore terminate at different distances from the skin surface, or at different distances from a microneedle mounting portion. In some embodiments, the two microneedles are different lengths. In other embodiments the microneedles are the same length, and a mounting portion is configured so as to axially displace one microneedle relative to the other. For example, the mounting portion may be multi-levelled with a first electrode extending from a first level and a second electrode extending from a second level.

For typical applications, the microneedles may extend outwardly from the apparatus for a distance of between about 10 μm and about 5000 μm. For many applications, distances between about 500 μm and about 4000 μm will be useful.

Those skilled in the art will appreciate that the invention described herein is susceptible to further variations and modifications other than those specifically described.

For example, the movable arm may be moved by the user squeezing or pressing on a flexible portion of the apparatus housing, by the actuation of a rotating lever, or by sliding an element along an inclined to urge the arm downward.

The skin contacting portion of the apparatus has been drawn as being strictly planar on its underside (the skin contacting surface), however in some embodiments it may be curved to conform to the surface of a bodily part such as the finger, wrist, heel, or ear. The skin contacting portion may have a degree of flexibility (in at least one direction) so as to be conformable to the surface of a bodily part.

The space through which a microneedle extends is generally shown as being an aperture, however other types of spaces are contemplated. In some embodiments the space is not an aperture, one such embodiment having microneedles extending through a space peripheral to the skin contact portion.

Those skilled in the art will appreciate that the invention described herein is susceptible to further variations and modifications other than those specifically described. It is understood that the invention comprises all such variations and modifications which fall within the spirit and scope of the present invention.

Accordingly, the spirit and scope of the present invention is not to be limited by the foregoing examples, but is to be understood in the broadest sense allowable by law.

1. A method for producing an electrochemical aptamer-based sensor apparatus, the method comprising assembling two or more electrodes of an electrochemical aptamer-based sensor with an apparatus for contacting the two or more electrodes to the skin of a subject.

2. The method of claim 1, wherein at least one of the two or more electrodes is a working electrode configured to specifically detect an analyte.

3. The method of claim 1, wherein at least two of the two or more electrodes is each a working electrode configured to specifically detect an analyte.

4. The method of claim 3, wherein each of the two or more working electrodes comprises a different aptamer species, each of the different aptamer species configured to specifically detect different analytes or the same analyte.

5. The method of claim 1, wherein the two or more electrodes are assembled in a fixed mutual spaced relationship.

6. (canceled)

7. (canceled)

8. (canceled)

9. (canceled)

10. (canceled)

11. (canceled)

12. (canceled)

13. (canceled)

14. (canceled)

15. The method of claim 1, wherein at least one of the electrodes is a wire, a needle, or a microneedle.

16. The method of claim 1, wherein the assembling comprises mounting each of the electrodes on a mounting portion.

17. The method of 16, wherein, when the sensor apparatus is assembled, one electrode terminates more distally to the mounting portion than the other.

18. (canceled)

19. The method of claim 16, wherein the mounting portion electrically insulates each electrode from each other electrode.

20. The method of claim 16, wherein the electrodes and/or the mounting portion are configured to form a watertight seal at a junction formed therebetween.

21. The method of claim 20, wherein the watertight seal is formed by way of a press fit, snap fit or friction fit between the electrode and the mounting portion; or by way of a flexible seal, or a curable sealant applied to or about the junction; or by way of a threaded connection between the electrode and the mounting portion.

22. (canceled)

23. (canceled)

24. The method of claim 16, wherein at least one of the electrodes comprises an expanded region configured to contact a surface of the mounting portion.

25. The method of claim 1, comprising assembling at least 3, 4, 5, 6, 7, 8, 9, 10, 20, 30, 40, 50, 60, 70, 80, 90, or 100 electrodes.

26. (canceled)

27. The method of claim 1, wherein each of the electrodes is obtained by removing an electrode from a group of electrodes of the same analyte specificity, dimension, material, or function.

28. The method of claim 27, wherein the group of electrodes is held in a holder configured to releasably hold the electrodes.

29. The method of claim 1, wherein at least one of the electrodes is a working electrode, and the working electrode(s) are selected from an electrode library comprising at least 2, 3, 4, 5, 6, 7, 8, 9, 10, 11, 12, 13, 14, 15, 16, 17, 18, 19, or 20 working electrodes each of which comprises a different aptamer species.

30. The method of claim 29, wherein working electrodes comprising the same aptamer species are grouped into a discrete holder, or grouped into a region of a single

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. A system for producing an electrochemical aptamer-based sensor apparatus, the system comprising:
a library of two or more working electrodes of an electrochemical aptamer-based sensor apparatus, and
a mounting portion configured to mount the two or more working electrodes in a fixed mutual spaced relationship, wherein each working electrode comprises a different aptamer species.

109. (canceled)

110. The system of claim 108, wherein the electrode library comprises working electrodes comprising at least 2, 3, 4, 5, 6, 7, 8, 9, 10, 11, 12, 13, 14, 15, 16, 17, 18, 19, or 20 different aptamer species.

111. The system of claim 110, wherein the working electrodes comprising the same aptamer species are grouped into a discrete holder, or grouped into a region of a single holder.
